(19) United States
(12) Patent Application Publication
(10) Pub. No.: US 2016/0376604 A1
(43) Pub. Date: Dec. 29, 2016

(54) SYSTEM FOR AUTOMATED EXPLANT PREPARATION AND METHOD OF USE

(71) Applicant: Dow AgroSciences LLC, Indianapolis, IN (US)

(72) Inventors: Donald L. McCarty, II, Freeland, MI (US); Siva Chennareddy, West Lafayette, IN (US); Toby Cicak, Indianapolis, IN (US); William E. Gee, Forest, VA (US); David Badour, Beaverton, MI (US); John Lund, Midland, MI (US); Rodrigo Sarria, West Lafayette, IN (US); Paul Morabito, Midland, MI (US)

(21) Appl. No.: 15/195,951

(22) Filed: Jun. 28, 2016

Related U.S. Application Data
(60) Provisional application No. 62/186,059, filed on Jun. 29, 2015.

(51) Int. Cl. C12N 15/82; C12M 1/12; C12M 1/00; C12M 1/22; C12M 3/06; C12M 3/00; C12M 1/36

(52) U.S. Cl. CPC C12N 15/8205; C12M 21/08; C12M 37/00; C12M 41/48; C12M 23/10; C12M 27/16; C12M 29/00

(57) ABSTRACT

A system and method for the automated or semi-automated preparation of explants for transformation and transgenic engineering.

### CROSS REFERENCE TO RELATED APPLICATIONS

This application claims the benefit under 25 USC §119(e) of U.S. Patent Application Ser. No. 62/186,059, filed on Jun. 29, 2015, the entire disclosure of which is incorporated herein by reference.

### TECHNICAL FIELD

The present disclosure relates generally to devices for preparing seeds for use in plant breeding, and, more specifically, to a device for preparing explants for gene transformation and transgenic engineering.

### BACKGROUND

Soybean (Glycine max) is one of the most important agricultural crops, with an annual crop yield of more than 200 million metric tons, and an estimated value exceeding 40 billion U.S. dollars worldwide. Soybean accounts for over 97% of all oilseed production globally. Thus, reliable and efficient methods for improving the quality and yield of this valuable crop are of significant interest.

Traditional breeding methods for improving soybean have been constrained because the majority of soybean cultivars are derived from only a few parental lines, leading to a narrow germplasm base for breeding. Christou et al., TIBTECH 8:145-151 (1990). Modern research efforts have focused on plant genetic engineering techniques to improve soybean production. Transgenic methods are designed to introduce desired genes into the heritable germline of crop plants to generate elite plant lines. The approach has successfully increased the resistance of several other crop plants to disease, insects, and herbicides, while improving nutritional value.

Several methods have been developed for transferring genes into plant tissue, including biolistics (such as high velocity microprojectile bombardment), microinjection, electroporation, and direct DNA uptake. Agrobacterium-mediated gene transformation has more recently been used to introduce genes of interest into soybeans. However, soybeans have proven to be a challenging system for transgenic engineering. Efficient transformation and regeneration of soybean explants is difficult to achieve, and frequently hard to repeat.

Agrobacterium tumefaciens, a pathogenic, soil-dwelling bacterium, has the inherent ability to transfer its DNA, called T-DNA, into host plant cells and to induce the host cells to produce metabolites useful for bacterial nutrition. Using recombinant techniques, some or all of the T-DNA may be replaced with a gene or genes of interest, creating a bacterial vector useful for transforming the host plant. Agrobacterium-mediated gene transfer is typically directed at undifferentiated cells in tissue culture, but may also be directed at differentiated cells taken from the leaf or stem of the plant. A number of procedures have been developed for Agrobacterium-mediated transformation of soybean, which may loosely be classified based on the explant tissue subjected to transformation.

U.S. Pat. No. 7,696,408, Olhoft, et al., discloses a cotyledonary node method for transforming both monocotyledonous and dicotyledonous plants. The “cot node” method involves removing the hypocotyl from 5-7 day old soybean seedlings by cutting just below the cotyledonary node, splitting and separating the remaining hypocotyl segment with the cotyledons, and removing the epicotyl from the cotyledon. The cotyledonary explant is wounded in the region of the axillary bud and/or cotyledonary node, and cultivated with Agrobacterium tumefaciens for five days in the dark. The method requires in-vitro germination of the seeds, and the wounding step introduces significant variability.

U.S. Pat. No. 6,384,301, Martinelli et al., discloses Agrobacterium-mediated gene delivery into living meristem tissue from soybean embryos excised from soybean seeds, followed by culturing of the meristem explant with a selection agent and hormone to induce shoot formation. Like the “cot node” method, the meristem explants are preferably wounded prior to infection.

U.S. Pat. No. 7,473,822, Paz et al., discloses a modified cotyledonary node method called the “half-seed explant” method. Mature soybean seeds are imbibed, surface-sterilized and split along the hilum. Prior to infection, the embryonic axis and shoots are completely removed, but no other wounding occurs. Agrobacterium-mediated transformation proceeds, potential transformants are selected, and explants are regenerated on selection medium.

Transformation efficiencies remain relatively low with these methods, on the order of 0.3% to 2.8% for the “cot node” method, 1.2 to 4.7% for the “meristem explant” method, and between 3.2% and 8.7% (overall 4.9%) for the “half-seed explant” method. Transformation efficiencies of approximately 3% are typical in the art.

An improved “split-seed” transgenic protocol may accelerate future production and development of transgenic soybean products. An efficient and high-throughput method for stable integration of a transgene into soybean tissue would facilitate breeding programs and have the potential to increase crop productivity.

### SUMMARY

A method and apparatuses for automated explant preparation are disclosed. According to one aspect, the automated explant preparation method may include operating a pump to fill an explant dish including a plurality of explants with an Agrobacterium solution, operating a first robotic arm to move the filled explant dish onto a shaker plate of a shaker station, operating the shaker station to move the shaker plate in a direction within a plane defined by the shaker plate to infect the plurality of explants with the Agrobacterium solution, and operating a second robotic arm to move an explant from the filled explant dish to a predetermined position on a cultivation media dish in response to determining the explant has been infected with the Agrobacterium solution.

In some embodiments, the method may further comprise operating the first robotic arm to move the cultivation media dish to the delivery station in response to determining the cultivation media dish has a predetermined number of explants positioned on the cultivation media dish. Further, in some embodiments, the method may further comprise determining the cultivation media dish has the predetermined number of explants positioned on the cultivation media dish comprises determining the cultivation media dish has a number (n) of explants positioned on the cultivation media dish and the explants are evenly spaced 360/n degrees apart on the cultivation media dish.

In some embodiments, operating the first robotic arm to move the cultivation media dish may comprise operating the first robotic arm to secure a lid of the cultivation media dish onto the cultivation media dish, and operating the first robotic arm to move the secured cultivation media dish to the delivery station.

In some embodiments, the method may further comprise capturing an image of a base of the filled explant dish with a camera, determining a location of an explant in the filled explant dish based on the image, and operating the second robotic arm to grip the explant at the location. Additionally, in some embodiments, operating the second robotic arm to move the explant may comprise operating the second robotic arm to move the explant in response to operating the second robotic arm to grip the explant.

In some embodiments, determining the location of the explant in the filled explant dish may comprise determining locations of the plurality of explants in the filled explant dish, and selecting the explant from the plurality of explants.

Additionally, in some embodiments, the method may further comprise selecting the cultivation media dish from a plurality of cultivation media dishes based on a number of explants currently positioned on each of the cultivation media dishes. In some embodiments, selecting the cultivation media dish may comprise selecting a cultivation media dish having fewer than six explants currently positioned on the cultivation media dish, and operating the second robotic arm to move the explant from the filled explant dish to the predetermined position on the selected cultivation media dish may comprise determining a predetermined position on the selected cultivation media dish to which to move the explant based on a position of each other explant currently positioned on the cultivation media dish.

In some embodiments, the method may further comprise operating the first robotic arm to move each cultivation media dish of a plurality of cultivation media dishes from a dish dispenser to a predetermined position on a transfer station different from a position of each other cultivation media dish of the plurality of cultivation media dishes. In some embodiments, the method may further comprise operating a second pump to pump the Agrobacterium solution from the filled explant dish and into a solution waste container in response to determining each cultivation media dish has a predetermined number of explants positioned on the cultivation media dish. Additionally, in some embodiments, the method may comprise operating the first robotic arm to move the filled explant dish to a dish waste container in response to determining the Agrobacterium solution has been removed from the filled explant dish.

In some embodiments, the method may comprise operating the first robotic arm to move the filled explant dish comprises operating a claw grip of the first robotic arm with a compressed air source to grasp the filled explant dish, and operating the second robotic arm to move the explant may comprise operating the second robotic arm to secure the explant with a suction force applied to the explant with a negative pressure source of the second robotic arm. In some embodiments, operating the second robotic arm to move the explant may comprise operating the second robotic arm to move the explant from the filled explant dish in response to determining that a desired infection time associated with infection of the explant has been reached.

In some embodiments, operating the shaker station to move the plate may comprise moving the plate in movement pattern including at least one of rotational or side-to-side movements within the plane defined by the plate. Further, in some embodiments, the method may comprise sterilizing a grip of the second robotic arm. In some embodiments, the Agrobacterium solution may comprise Agrobacterium tumefaciens. In other embodiments, the Agrobacterium solution may comprise Agrobacterium rhizogenes. Additionally, in some embodiments, the explants may comprise soybean explants. In other embodiments, the explants may comprise canola hypocotyl segments.

According to another aspect, an explant preparation apparatus may include a first robotic arm including a claw grip to grasp explant dishes for movement, a second robotic arm including a suction grip to secure explants with suction force for movement, a pump configured to deliver an Agrobacterium solution, a shaker station including a shaker plate and configured to move the shaker plate, and an electronic controller. In some embodiments, the electronic controller may be configured to operate the pump to fill an explant dish including a plurality of explants with an Agrobacterium solution, operate the first robotic arm to move the filled explant dish onto a shaker plate of a shaker station, operate the shaker station to move the shaker plate in a direction within a plane defined by the shaker plate to infect the plurality of explants with the Agrobacterium solution, and operate the second robotic arm to move an explant from the filled explant dish to a predetermined position on a cultivation media dish in response to a determination that the explant has been infected with the Agrobacterium solution.

In some embodiments, the explant preparation apparatus may further comprise a third robotic arm including a claw grip to grasp explant dishes for movement. In some embodiments, the first robotic arm may include a compressed air source, and the electronic controller may be configured to operate the compressed air source to move the claw grip between an open and closed position. In some embodiments, the Agrobacterium solution may comprise Agrobacterium tumefaciens. In other embodiments, the Agrobacterium solution may comprise Agrobacterium rhizogenes. Additionally, in some embodiments, the explants may comprise soybean explants. In other embodiments, the explants may comprise canola hypocotyl segments.

According to yet another aspect, a dish dispensing system may comprise a housing, an elongated body secured to the housing and centered about a longitudinal axis, wherein the elongated body is configured to secure a stack of petri dishes along the longitudinal axis, a first pneumatic device positioned in the housing and configured to move a set of petri dishes of the stack of petri dishes along the longitudinal axis in a first direction to separate a first petri dish of the stack of petri dishes from the set of petri dishes, and a second pneumatic device positioned in the housing and configured to move the separated first petri dish along an axis orthogonal to the longitudinal axis.

In some embodiments, the first pneumatic device may comprise a pair of dish gripping arms configured to secure a bottom petri dish of the set of petri dishes. Additionally, in some embodiments, the second pneumatic device may be configured to move the separated first petri dish to a location outside the housing. In some embodiments, the dish dispensing system may further comprise a third pneumatic device positioned in the housing and configured to move the set of petri dishes in a second direction opposite the first direction in response to a determination that the separated first petri dish has been removed from a plate extender operated by the second pneumatic device.

### BRIEF DESCRIPTION OF THE DRAWINGS

The detailed description particularly refers to the following figures, in which:

FIGS. 1 and 2 are perspective views of a system for preparing explants, e.g., soybean seed explants for gene transformation;

FIG. 3 is a top plan view of the system of FIG. 1;

FIG. 4 is a side elevation view of a claw grip assembly of a robotic arm of the system of FIG. 1;

FIG. 5 is a perspective view of a claw grip of the claw grip assembly of FIG. 4;

FIG. 6 is a perspective view of a suction grip assembly of a robotic arm of the system of FIG. 1;

FIG. 7 is a perspective view of a pumping system of the system of FIG. 1;

FIG. 8 is a perspective view of a partially assembled fluid delivery system of the pumping system of FIG. 7;

FIG. 9 is a side elevation view of the assembled fluid delivery system of FIG. 8 in use;

FIG. 10 is a side elevation view of a fluid extraction system of the pumping system of FIG. 7 in use;

FIG. 11 is a perspective view of a shaker station of the system of FIG. 1;

FIGS. 12-19 are views of a dish dispensing system of the system of FIG. 1 in various operational states;

FIG. 20 is a perspective view of a delivery station of the system of FIG. 1;

FIG. 21 is a perspective view of a sterilizing device of the system of FIG. 1;

FIG. 22 is a perspective view of a transfer station of the system of FIG. 1 including an imaging station;

FIG. 23 is a simplified block diagram of the system of FIG. 1;

FIGS. 24-25 are block diagrams showing an illustrative operating procedure for the system of FIG. 1;

FIG. 26 is a block diagram showing an illustrative procedure for moving cultivation media dishes from the dish dispensing system of FIGS. 12-19 to a transfer station of the system of FIG. 1;

FIG. 27 is a block diagram showing an illustrative procedure for moving infected explants from a filled explant dish to cultivation media dishes;

FIG. 28 is a block diagram showing an illustrative procedure for moving cultivation media dishes to the delivery station of FIG. 20; and

FIGS. 29-30 are illustrations of an image capture process of the operating procedure of FIG. 27 to identify an explant to be picked up by the system of FIG. 1.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

As used herein, the terms “grasping” and “gripping” refers to holding or seizing an explant, e.g., soybean seed explant or canola hypocotyl segment, with a tool. Any subsequent mechanism or action that allows the explant to be firmly clasped is considered within the scope of the term grasping.

As used herein, the term “genetically modified” or “transgenic” plant refers to a plant cell, plant tissue, plant part, plant germplasm, or plant which comprises a preselected DNA sequence which is introduced into the genome of a plant cell, plant tissue, plant part, plant germplasm, or plant by transformation.

As used herein, the term “transgenic,” “heterologous,” “introduced,” or “foreign” DNA or gene refer to a recombinant DNA sequence or gene that does not naturally occur in the genome of the plant that is the recipient of the recombinant DNA or gene, or that occurs in the recipient plant at a different location or association in the genome than in the untransformed plant.

As used herein, the term “explant” refers to a piece of plant tissue, e.g., transformable plant tissue, such as soybean explant tissue or canola hypocotyl, that is removed or isolated from a donor plant (e.g., from a donor seed), cultured in vitro, and is capable of growth in a suitable media.

As used herein, the term “plant” refers to either a whole plant, plant tissue, plant part, including pollen, seeds, or an embryo, plant germplasm, plant cell, or group of plants. The class of plants that can be used in the method of the invention is not limited to soybeans, but may generally include any plants that are amenable to transformation techniques, including both monocotyledonous and dicotyledonous plants.

As used herein the term “transformation” refers to the transfer and integration of a nucleic acid or fragment into a host organism, resulting in genetically stable inheritance. Host organisms containing the transformed nucleic acid fragments are referred to as “transgenic” or “recombinant” or “transformed” organisms. Known methods of transformation include Agrobacterium tumefaciens or Agrobacterium rhizogenes mediated transformation, calcium phosphate transformation, polybrene transformation, protoplast fusion, electroporation, ultrasonic methods (e.g., sonoporation), liposome transformation, microinjection, naked DNA, plasmid vectors, viral vectors, biolistics (microparticle bombardment), silicon carbide WHISKERS™ mediated transformation, aerosol beaming, or PEG transformation as well as other possible methods.

As used herein, “transformable plant tissue” refers to any plant part suitable for transformation by Agrobacterium, which has a wide host range in plants. Nester E., Front Plant Sci. 5:730 (2015). Transformable plant tissues include cells from dicot or monocot plant species, such as for example, soybean (Glycine max); rapeseed (also described as canola) (Brassica napus); maize (also described as corn) (Zea mays); cotton (Gossypium spp.); safflower (Carthamus tinctorius); sunflower (Helianthus annuus); tobacco (Nicotiana tabacum); Arabidopsis thaliana; castor bean (Ricinus communis); coconut (Cocus nucifera); coriander (Coriandrum sativum); groundnut (Arachis hypogaea); oil palm (Elaeis guineeis); olive (Olea eurpaea); rice (Oryza sativa); squash (Cucurbita maxima); barley (Hordeum vulgare); sugarcane (Saccharum officinarum); rice (Oryza sativa); wheat (Triticum spp. including Triticum durum and Triticum aestivum); duckweed (Lemnaceae sp.); sugarbeet (Beta vulgaris); alfalfa (Medicago sativa); sorghum; and turf grasses. Thus, any suitable plant species or plant cell can be selected as a source of transformable plant tissue. In some embodiments, transformable plant tissues include pollen, embryos, flowers, fruits, shoots, leaves, roots, stems, and explants.

Transformable plant tissues which can be used to regenerate a plant include tissues from, for example, embryos, immature embryos, hypocotyl cells (e.g., canola hypocotyl segments), meristematic cells, callus, pollen, leaves, anthers, roots, root tips, silk, flowers, and kernels. Transformable plant tissue also includes protoplasts and spheroplasts, which refer to plant cells having their cell wall completely and partially removed.

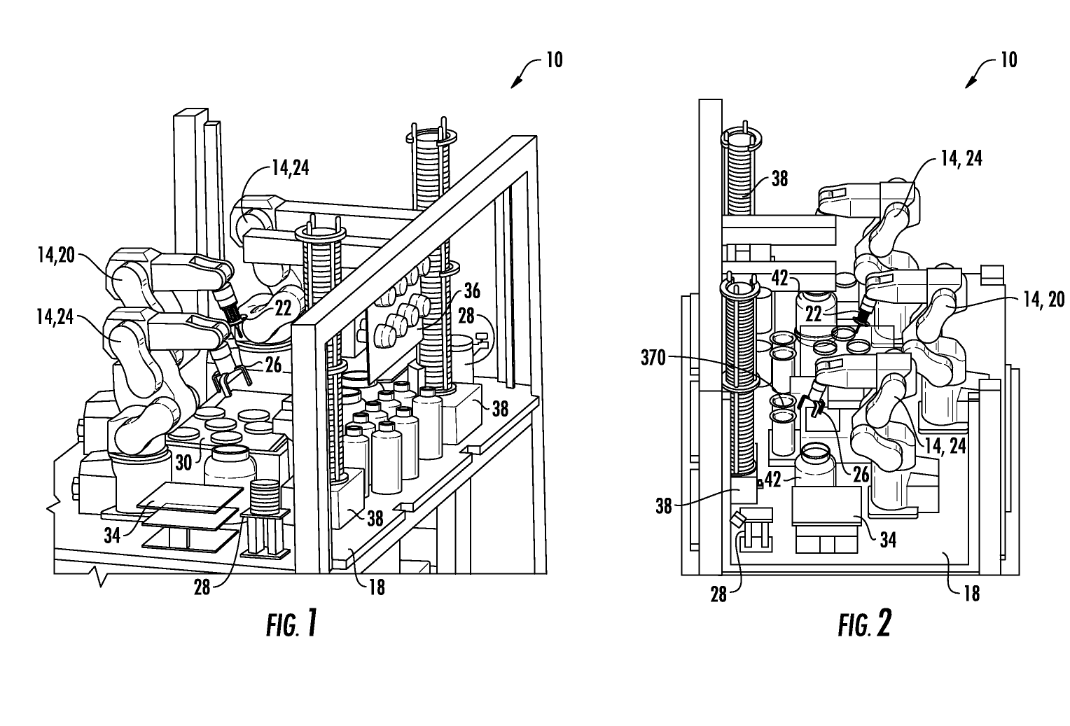

Referring to FIGS. 1-3, a system 10 for automated preparation of explants, for example, soybean seed explants or canola hypocotyl segments, for gene transformation by any known method is shown. The system 10 is illustratively configured to prepare soybean seed explants (hereinafter seed explants 12) as part of a transgenic protocol and the development of transgenic soybean products. Exemplary transgenic protocols are described in U.S. patent application Ser. No. 14/133,370 entitled “IMPROVED SOYBEAN TRANSFORMATION FOR EFFICIENT AND HIGH-THROUGHPUT TRANSGENIC EVENT PRODUCTION” and U.S. patent application Ser. No. 14/134,883 entitled “IMPROVED SOYBEAN TRANSFORMATION FOR EFFICIENT AND HIGH-THROUGHPUT TRANSGENIC EVENT PRODUCTION,” which are expressly incorporated herein by reference. Further, in some embodiments, the techniques described herein may be employed in conjunction with the techniques described in U.S. Provisional Patent Application No. 61/989,266 entitled “SYSTEM FOR IMAGING AND ORIENTING SEEDS AND METHOD OF USE,” U.S. Provisional Patent Application No. 61/989,275 entitled “SYSTEM FOR SEED PREPARATION AND METHOD OF USE,” and/or U.S. Provisional Patent Application No. 61/989,276 entitled “SYSTEM FOR CUTTING AND PREPARING SEEDS AND METHOD OF USE,” which are expressly incorporated herein by reference.

More specifically, as described below, the system 10 is configured to deliver an Agrobacterium solution, containing Agrobacterium tumefaciens or Agrobacterium rhizogenes, to a dish of explants, e.g., seed explants or hypocotyl segments 12, agitate the explants 12 (e.g., by shaking the dish of seed explants 12), and transfer the explants 12 to a cultivation media dish (e.g., a dish of agar growing media). The system 10 reduces a risk of injury to a user associated with repetition of the tasks involved in the procedure, reduces personnel exposure to Agrobacterium solutions, and ensures that the explants 12 are treated equally for quality assurance.

It should be appreciated that any of the devices and methods described herein can be used in connection with the transformation methods disclosed in those applications. It should also be appreciated that in other embodiments any of the devices and methods described herein may be configured for use with other classes of plants that are amenable to transformation techniques, including both monocotyledonous and dicotyledonous plants.

The system 10 includes a set of robotic arms 14 that move the explants 12 and/or dishes 16 between various stations arranged on a table 18. In the illustrative embodiment, each robotic arm 14 is an Epson model C3 six-axis articulated arm that is configured to operate independently of each other robotic arm 14. In other embodiments, the robotic arm 14 may have a different number of degrees of freedom than those descried herein. For example, the robotic arms 14 may be embodied as robotic arms having at least two independent axes.

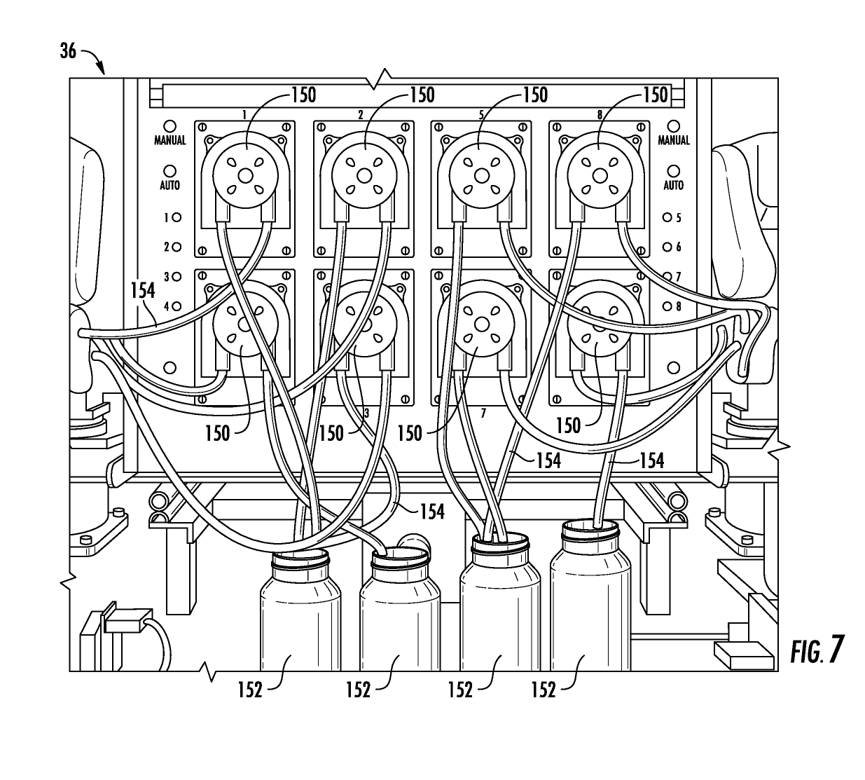

In the illustrative embodiment, one of the robotic arms 14 (hereinafter robotic arm 20) includes a suction grip 22 configured to grasp and hold an explant 12 (see FIG. 6), and each of the other robotic arms 14 (hereinafter robotic arms 24) includes a claw grip 26 configured to grasp and hold a dish 16 or a portion of a dish 16 (e.g., the base of the dish 16 or the lid of the dish 16). In some embodiments, the system 10 may operate with one of the robotic arms 24 out-of-service. Further, it should be appreciated that in other embodiments, the system 10 may include only a single robotic arm 24 to move the dishes 16 between the various stations arranged on the table 18. Additionally, in the illustrative embodiment, each robotic arm 14 is capable of rotating the corresponding grip 22, 26 about its axis by at least 180 degrees.

As shown in FIGS. 1-3, the stations arranged on the table 18 include a pair of delivery stations 28 and a pair of transfer stations 30. In the illustrative embodiment, the delivery stations 28 are positioned at the back of the table 18 toward opposite ends of the table 18 where dishes 16 may be positioned by a user for processing by the system 10 and where dishes 16 may be positioned for retrieval by the user subsequent to the processing by the system 10. Further, the transfer stations 30 are positioned toward the middle of the table 18 such that each of the transfer stations 30 is accessible by the robotic arm 20 and at least one of the robotic arms 24. As described in greater detail below, the transfer stations 30 are used to transfer explants 12 infected with an Agrobacterium tumefaciens solution to dishes 16 including cultivation media (e.g., agar).

Each of the transfer stations 30 also includes an imaging station 32 that is operable to capture a number of images of the explants 12 within a dish 16. The system 10 also includes a pair of shaker stations 34 that are operable to agitate or shake explants 12 within dishes 16 containing an Agrobacterium solution containing Agrobacterium tumefaciens or Agrobacterium rhizogenes. The system 10 also includes a pumping system 36 that is configured to deliver an Agrobacterium solution to the dishes 16 and to extract the Agrobacterium solution from the dishes 16 subsequent to infection of the explants 12 included in those dishes 16 by the Agrobacterium solution. Additionally, the system 10 includes a pair of dish dispensing systems 38 configured to hold and to dispense dishes 16 for use by the system 10. In the illustrative embodiment, each of the dish dispensing systems 38 is configured to hold as many as fifty dishes at a given time. The system 10 also includes a sterilization device 40 that is configured to sterilize the suction grip 22 of the robotic arm 20 and a pair of dish waste containers 42 that receive discarded dishes 16 subsequent to use by the system 10.

In use, the system 10 may be operated to infect automatically a number of explants 12 for transformation. To do so, the system 10 may pump the Agrobacterium solution into a dish 16 of explants 12. The system 10 may then operate a robotic arm 24 to place the Agrobacterium-filled dish 16 of explants 12 onto the shaker station 34 for a predetermined amount of time (e.g., thirty minutes) to ensure the explants 12 are properly infected. While the explants 12 are at the shaker station 34, the system 10 may operate the robotic arm 24 to dispense dishes 16 containing cultivation media such as agar at predetermined locations on the transfer station 30. After the predetermined amount of time at the shaker station 34 has elapsed, the system 10 may operate the robotic arm 24 to move the Agrobacterium-filled dish 16 of infected explants 12 onto the imaging station 32. One or more images of the Agrobacterium-filled dish 16 may be captured at the imaging station 32 to determine the locations of the explants 12 on the dish 16. Based on the captured image(s), the system 10 may operate the robotic arm 20 to grasp the explants 12 individually from the dish 16 and to move each of the explants 12 to predetermined locations within the cultivation media dishes 16. After the cultivation media dishes 16 have been filled with the predetermined number of infected explants 12, the system 10 may operate the robotic arm 24 to move each of the filled cultivation media dishes 16 to the corresponding delivery station 28 at which the user of the system 10 may retrieve those cultivation media dishes 16. The system 10 may operate the robotic arm 24 to move the Agrobacterium-filled dish 16 to the pumping system 36 for extraction of the Agrobacterium solution and discard the empty dish 16 in the dish waste container 42. Each of these processing steps and the various components of the system 10 are described in greater detail below in references to FIGS. 4-30.

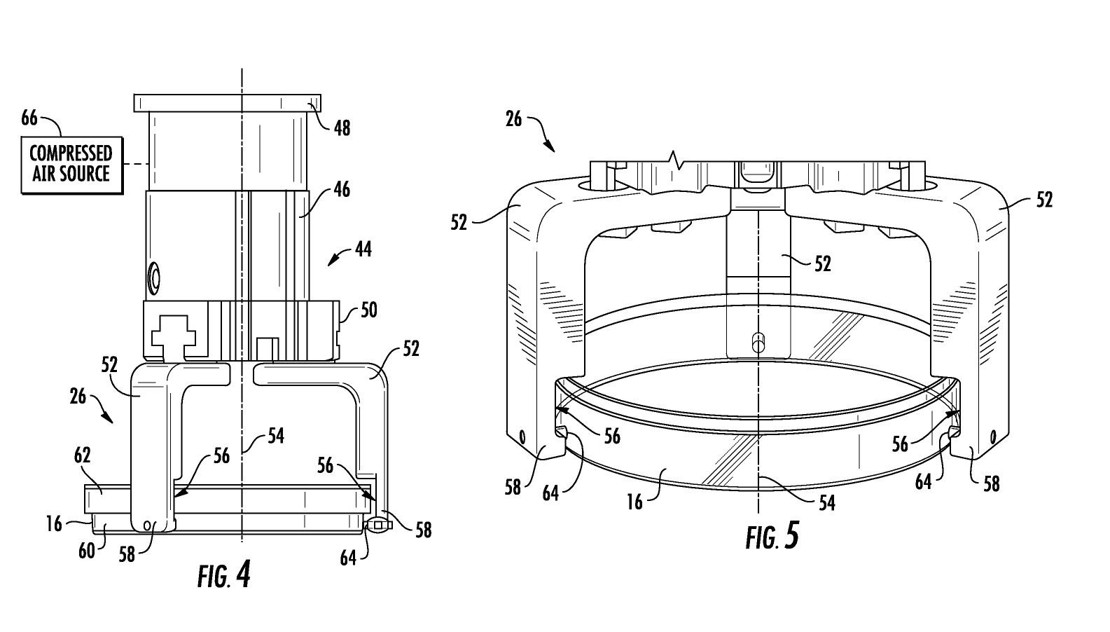

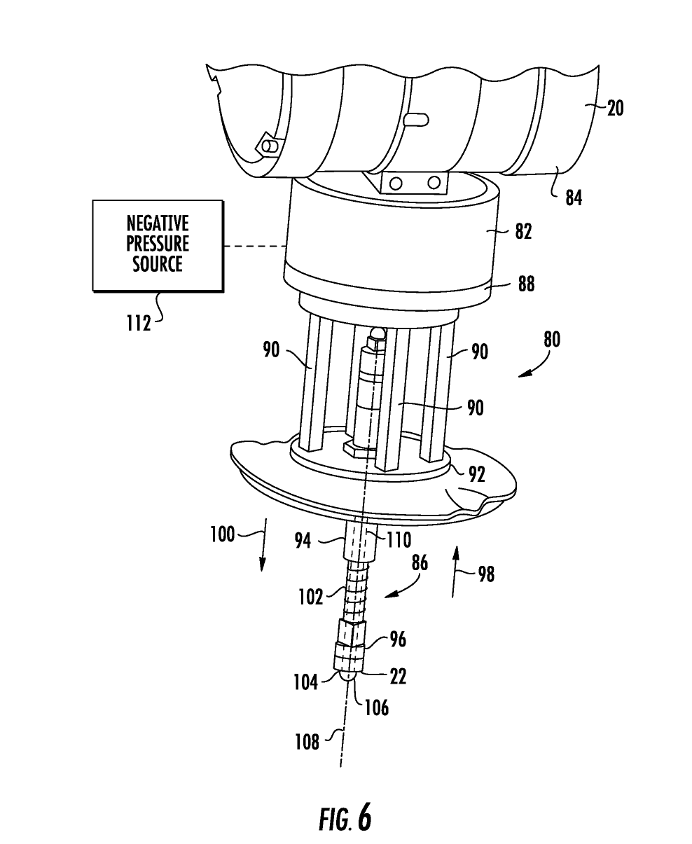

Referring now to FIGS. 4-5, a portion of one of the robotic arms 24 including the claw grip 26 is shown in greater detail. In the illustrative embodiment, each of the robotic arms 24 includes a grip assembly 44 configured to grasp and hold a dish 16 or a portion of a dish 16 (e.g., the base of the dish 16 or the lid of the dish 16). In the illustrative embodiment, the grip assembly 44 includes a body 46 that is attached to a distal section 48 of each arm 24. The claw grip 26 is secured to a distal section 50 of the body 46.

The illustrative claw grip 26 of the grip assembly 44 includes three fingers 52 that are configured to move radially inward and outward from a longitudinal axis 54 defined along the grip assembly 44 such that the claw grip 26 or, more particularly, the fingers 52 may be advanced into contact with and out of contact with a dish 16. In the illustrative embodiment, the fingers 52 of the claw grip 26 are evenly spaced apart such that each of the fingers 52 is spaced apart approximately 120 degrees about the longitudinal axis 54 from each of the other fingers 52.

As shown, each of the fingers 52 extends distally from the distal section 50 of the body 46 of the grip assembly 44. Further, each of the fingers 52 includes an aperture 56 defined in a distal end 58 of the corresponding finger 52. Further, a contact screw 64 extends through the distal end 58 of the finger 52 in a direction radial to the longitudinal axis 54 and is configured to contact the dish 16 to grasp the dish 16. It should be appreciated that each of the dishes 16 being used herein may be embodied as a petri dish or any other dish that includes a base 60 and a lid 62 that rests on the top of and overlaps the base 60 when the lid 62 is secured to the base 60.

As shown in FIG. 4, the grip assembly 44 is configured to grasp and hold the dish 16 by moving the fingers 52 of the claw grip 26 into contact with the dish 16. When a dish 16 is grasped by the claw grip 26, the lid 62 (if not already removed) is configured to rest in the aperture 56 and the contact screw 64 is configured to contact the base 60. Further, the robotic arm 24 may operate the grip assembly 44 to remove the lid 62 of the dish 16 by moving the fingers 52 into a position just short of contacting the base 60 of the dish 16 and then move the grip assembly 44 in a direction along the longitudinal axis 54 away from the base 60.

In the illustrative embodiment, the robotic arm 24 includes a compressed air source 66 (e.g., a compressed air pump), which is configured to regulate the pressure of compressed air supplied to the grip assembly 44 and to the claw grip 26. In the illustrative embodiment, when grasping a dish 16, the compressed air source 66 is configured to supply enough pressure to hold the dish 16 securely without crushing the dish 16, which may be fragile. In the illustrative embodiment, the body 46 of the grip assembly 44 is embodied as a three-finger, 32 mm bore gripper, part number MHSL3-32D with type D-Y59AZ positioning grippers, which is commercially available from SMC Pneumatics.

Referring now to FIG. 6, the robotic arm 20 of the system 10 includes a grip assembly 80 configured to grasp and hold an explant 12. In the illustrative embodiment, the grip assembly 80 includes a body 82 that is attached to a distal section 84 of the robotic arm 20. The grip assembly 80 also includes a suspension mechanism 86 that connects the body 82 to a suction grip 22. The body 82 has a proximal disk 88 that is secured to the distal section 84 and a plurality of posts 90 that extend from the proximal disk 88 to a distal disk 92.

The suspension mechanism 86 extends from a proximal end 94 that is secured to the disk 92 to a distal end 96. As shown in FIG. 13, the grip 22 is secured to the distal end 96 of the suspension mechanism 86. The suspension mechanism 86 is configured to permit some axial movement of the grip 22, as indicated by arrows 98, 100, such that the grip 22 may be advanced into contact with an explant 12 without crushing the explant. In the illustrative embodiment, the suspension mechanism 86 includes a biasing element such as, for example, a helical spring 102, that biases the grip 22 outward, in the direction indicated by arrow 100.

The grip 22 of the assembly 80 is configured to grasp and hold an explant 12. In the illustrative embodiment, the grip 22 includes a cylindrical body 104 that is secured to the distal end 96 of the suspension mechanism 86. The body 104 is formed from an elastomeric material such as, for example, Viton, which is commercially available from DuPont Corporation. It should be appreciated that in other embodiments other elastomeric materials may be used. The body 104 includes a bellows, which provides the body 104 with limited flexibility. The body 104 also has a high temperature rating to permit sterilization of the grip 22. In the illustrative embodiment, the temperature rating is 446 degrees Fahrenheit. It should be appreciated that in other embodiments other elastomeric materials may be used.

The grip assembly 80 is configured to grasp and hold the explant 12 via vacuum. To do so, the grip 22 includes a hollow passageway 106 that extends longitudinally through the body 104 along an axis 108. The passageway 106 is connected to passageways 110 defined in the suspension mechanism 86 and the body 82 of the grip assembly 80 and a negative pressure source 112. The negative pressure source 112 is illustratively embodied as a pump and is electrically coupled to a controller 500. The controller 500 may operate the source 112 to draw a vacuum through the passageways 106, 110 and secure an explant 12 to the grip 22. In the illustrative embodiment, the grip 22 has a radius of less than fifty percent of the average length of an explant 12, which may vary depending on, for example, the particular species of the seed explant 12.

Referring now to FIGS. 7-10, the pumping system 36 includes a plurality of pumps 150, each of which is configured to deliver an Agrobacterium solution containing Agrobacterium tumefaciens or Agrobacterium rhizogenes to a dish 16 or extract the Agrobacterium solution from a dish 16 (e.g., subsequent to infection of the explants 12 in the dish 16). As shown, in the illustrative embodiment, the pumping system 36 includes eight pumps 150 from which six of the pumps 150 are used to deliver the Agrobacterium solution (i.e., outflow pumps) and two of the pumps 150 are used to siphon/remove the Agrobacterium solution (i.e., inflow pumps). In some embodiments, the pumps 150 may be wired to rotate in only one direction, which prevents the pumps 150 that are used for siphoning from inadvertently running in reverse and spilled used Agrobacterium solution onto the table 18. In the illustrative embodiment, pump tubing 154 connects the pumps 150 to solution containers 152 that store Agrobacterium, some of which may be used to store unused Agrobacterium and the remainder of which may be used to store the used Agrobacterium. In particular, in use, a particular pump 150 extracts unused Agrobacterium from one of the solution containers 152 and delivers the extracted solution to a dish 16. Subsequent to use, another pump 150 may extract the used Agrobacterium solution from the dish 16 and dispense the used solution in another one of the solution containers 152. In some embodiments, each of the pumps 150 may be embodied as a peristaltic pump (e.g., a peristaltic pump commercially available from Welco, Co., Ltd.). Further, the pump tubing 154 may be embodied as 3/16^{th} inch PharMed® BPT peristaltic pump tubing commercially available from Thermo Fisher Scientific, Inc.

The illustrative pumping system 36 includes a fluid delivery station 160 at which the pumping system 36 is configured to pump fluid into a vessel (e.g., a dish 16 of seed explants 12) and a fluid extraction station 162 at which the pumping system 36 is configured to extract fluid from a vessel (e.g., a dish 16 of used Agrobacterium solution). In the illustrative embodiment, the pumping system 36 includes a separate fluid delivery station 160 for each of the robotic arms 24 and a separate fluid extraction station 162 for each of the arms 24 (e.g., positioned at opposite ends of the pumping system 36).

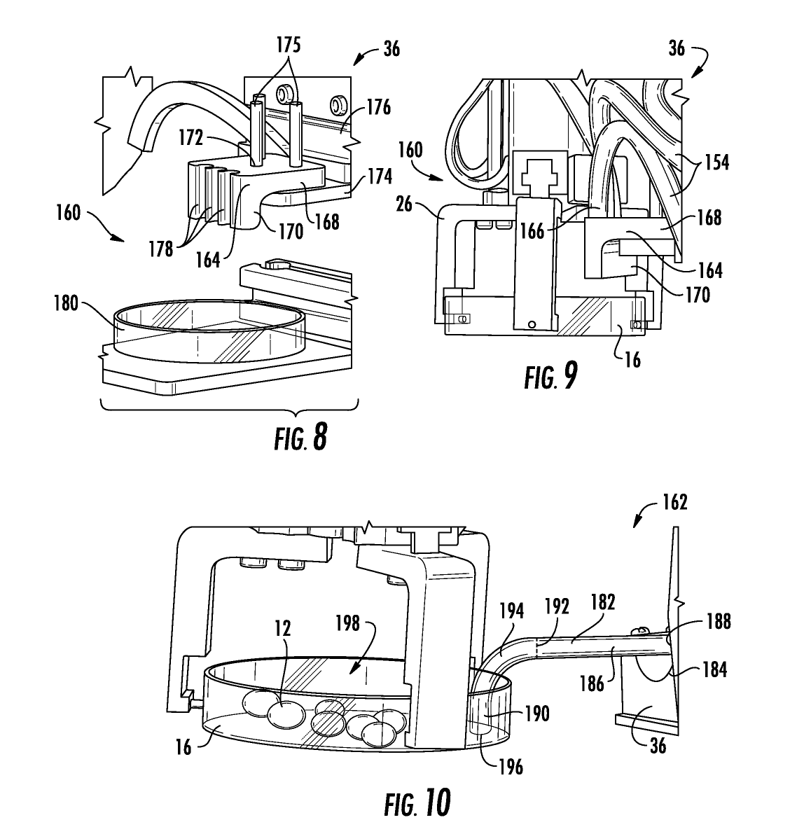

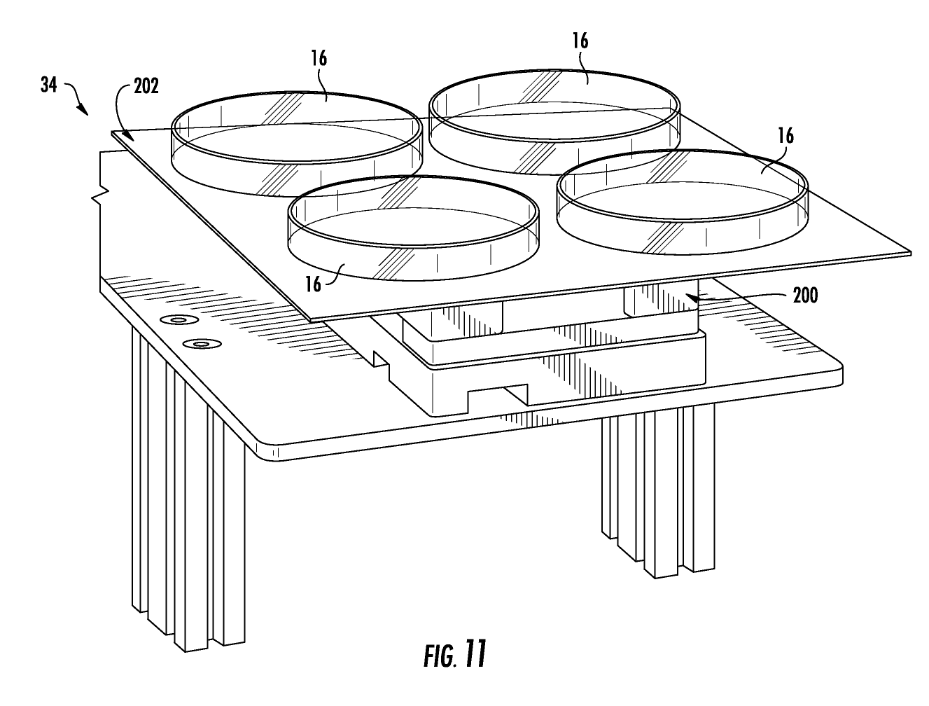

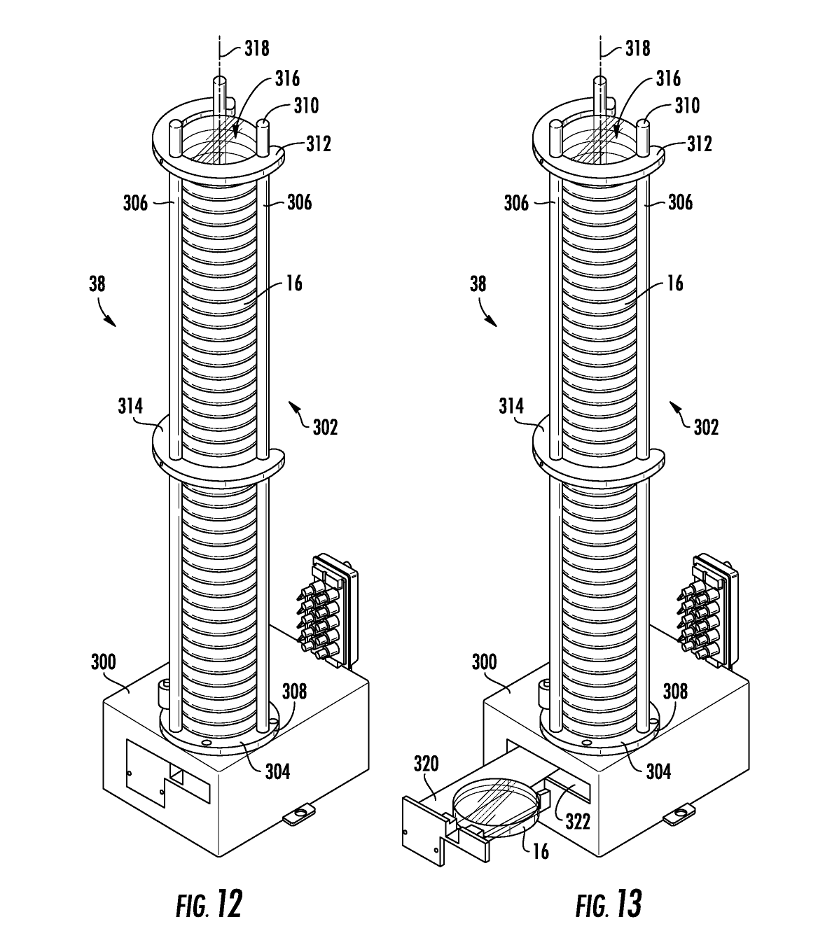

As shown in FIGS. 8-9, the fluid delivery station 160 includes a tube holder 164 that is configured to secure an end 166 of the pump tubing 154 to control the direction of flow of fluid from the pumping system 36 when pumping the fluid into a dish 16. It should be appreciated that the pump tubing 154 extends from the end 166 at the tube holder 164 to the corresponding pump 150. In the illustrative embodiment, the tube holder 164 includes a base 168 and tube section 170 extending generally perpendicularly from the base 168. The base 168 of the tube holder 164 includes a plurality of apertures 172 defined therein that define passageways through the base 168. As shown, the fluid delivery station 160 of the pumping system 36 includes a horizontal plate 174 that extends outward in a horizontal direction from a base 176 of the fluid delivery station 160. The fluid delivery station 160 also includes a corresponding plurality of posts 175 that extend upward perpendicularly from the horizontal plate 174 and are configured to be received in the plurality of apertures 172 of the base 168. In the illustrative embodiment, the fluid delivery station 160 includes a set of three apertures 172 and a corresponding set of three posts 175; however, in other embodiments, the fluid delivery station 160 may include a different number of posts 175 and/or apertures 172.

The tube section 170 of the tube holder 164 includes a plurality of grooves 178 defined therein that extend perpendicularly from the base 168 and are designed to secure the pump tubing 154. That is, in the illustrative embodiment, each piece of the pump tubing 154 used for fluid delivery is configured to pass through and be maintained securely within a passageway defined by the corresponding groove 178. Further, in the illustrative embodiment, the fluid delivery station 160 includes a drip pan 180 (e.g., an empty dish 16) positioned below the tube holder 164 and configured to contain any fluid that inadvertently drips from the end 166 of the pump tubing 154 at the tube holder 164. As shown in FIG. 9, in use, the robotic arm 24 controls the claw grip 26 to secure a dish 16 and move the dish 16 to a position below the end 166 of the pump tubing 154. After the dish 16 is properly positioned, the pumping system 36 may operating the corresponding pump(s) 150 to deliver fluid (e.g., an Agrobacterium solution) to the dish 16.

As shown in FIG. 10, the fluid extraction station 162 includes an extraction tube 182 that extends from a base 184 of the fluid extraction station 162 and is configured to extract fluid from a vessel (e.g., a dish 16). The extraction tube 182 includes a first straight section 186 coupled to pump tubing 154 at a first end 188 that extends into a solution container 152 for disposal of fluid extracted by the fluid extraction station 162. The extraction tube 182 also includes a second straight section 190 that is connected to a second end 192 of the first straight section 186 by a curved section 194. In the illustrative embodiment, the curved section 194 is embodied as a 90-degree interconnection such that the first straight section 186 and the second straight section 190 are approximately perpendicular to one another. In the illustrative embodiment, the extraction tube 182 is embodied as a 6-inch section of hollow ¼-inch stainless steel tubing with a 90-degree bend at a small aperture defined in the end of the tubing to prevent suctioning of the dish 16. However, the extraction tube 182 may be otherwise constructed in other embodiments. In use, the robotic arm 24, may control the claw grip 26 to secure a dish 16 and move the dish 16 to a position in which a distal end 196 of the extraction tube 182 is positioned within a bin 198 of the dish 16. It should be appreciated that each of the dishes 16 includes a bin 198 that receives the explants 12 and/or the Agrobacterium solution. In some embodiments, the dish 16 may be embodied as a petri dish. In operation, moving the dish 16 such that the extraction tube 182 is inserted in the bin 198, the robotic arm 24 may tilt the dish 16 toward the extraction tube 182 in order to force the fluid toward the end 196 of the extraction tube 182. The pumping system 36 may operate the corresponding pump 150 to extract the fluid (e.g., the Agrobacterium solution) from the dish 16.

As discussed above, the illustrative system 10 includes a pair of shaker stations 34 that are operable to agitate or shake explants 12 within dishes 16 containing an Agrobacterium tumefaciens solution. In particular, in the illustrative embodiment, one of the shaker stations 34 may be reached by one of the robotic arms 24, and the other shaker station 34 may be reached by the other robotic arm 24 (see FIG. 3). As shown in FIG. 11, each of the illustrative shaker stations 34 includes drive stage 200 and a shaker plate 202 coupled to the drive stage 200. In the illustrative embodiment, the drive stage 200 is configured to move the shaker plate 202 within a plane defined by the shaker plate 202 in order to agitate the contents of dishes 16 positioned on the shaker plate 202. In particular, in the illustrative embodiment, the shaker station 34 shakes up to four dishes 16 of explants 12 in the Agrobacterium solution for 30 minutes. In other embodiments, the explants 12 may be exposed and/or mixed with the Agrobacterium solution for a different period of time.

It should be appreciated that the illustrative drive stage 200 includes an electric motor (not shown) that is electrically connected to the controller, described below, and is operable to move the shaker plate 202 in a rotational, side-to-side, and/or other type of motion within the plane defined by the shaker plate 202. In some embodiments, the shaker station 34 may include a drive stage, model T-LSM025B, which is commercially available from Zaber Technologies, Inc. or a Variomag Teleshake unit, which is commercially available from Thermo Fisher Scientific, Inc. Further, depending on the particular embodiment, the shaker plate 202 may be constructed of aluminum, Plexiglas, Teflon, and/or another suitable material.

As described above, the illustrative system 10 includes a pair of dish dispensing systems 38 configured to hold and to dispense dishes 16 for use by the system 10. In particular, in the illustrative embodiment, the dish dispensing systems 38 may dispense dishes 16 that are filled with cultivation media (e.g., agar). Referring now to FIGS. 12-19, one of the dish dispensing systems 38 and its operation are shown. As shown in FIG. 12, the dish dispensing system 38 includes a housing 300 and an elongated body 302 that is secured to and extends upwardly from the housing 300. The elongated body 302 includes a curved base plate 304 that is secured to the housing 300 and a plurality of posts 306, each of which is secured to the curved base plate 304 at a proximal end 308 of the post 306 and extending upwardly from the curved base plate 304 to a distal end 310. The posts 306 are secured by a curved plate 312 at the distal end 310 and by another curved plate 314 at a point between the proximal end 308 and the distal end 310 of the posts 306. As such, in the illustrative embodiments, the dish dispensing system 38 includes three posts 306 that are secured at three points so as to prevent the posts 306 from moving or warping. In other embodiments, the dish dispensing system 38 may include a different number of posts 306 and/or support points.

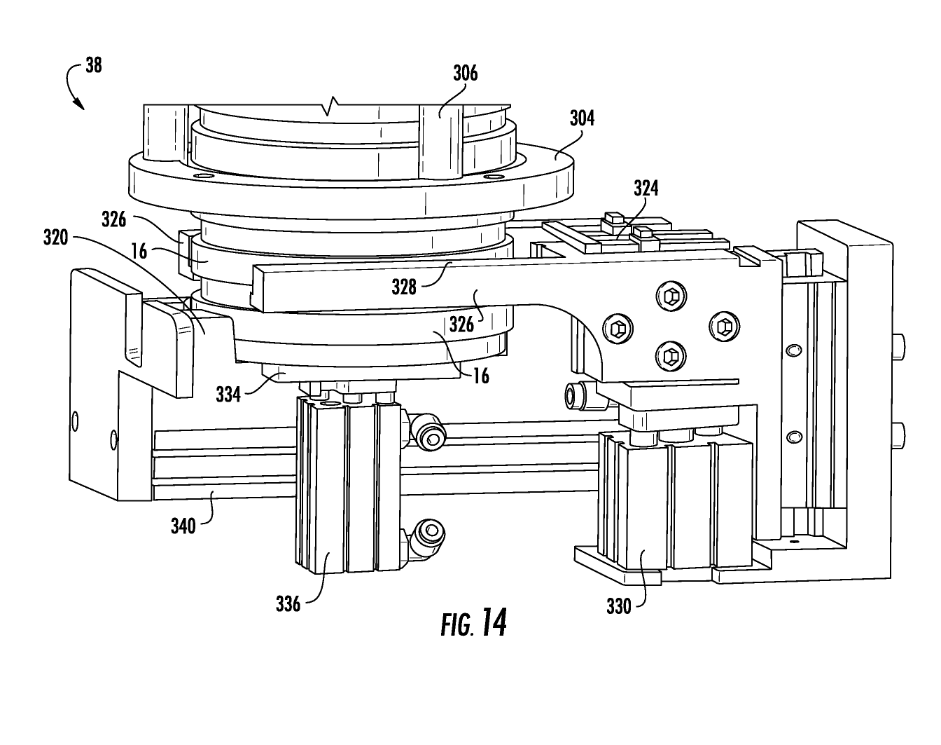

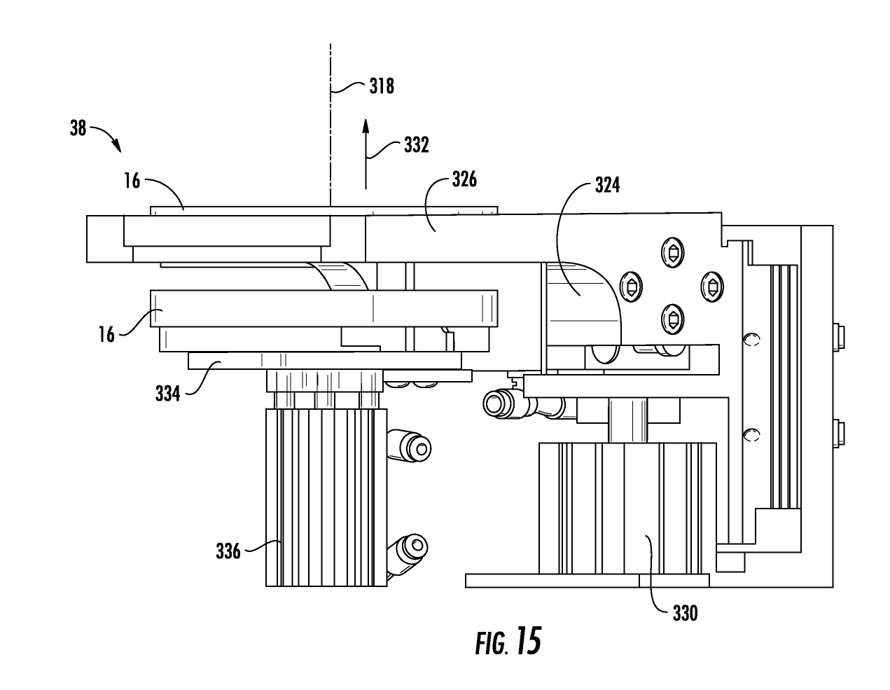

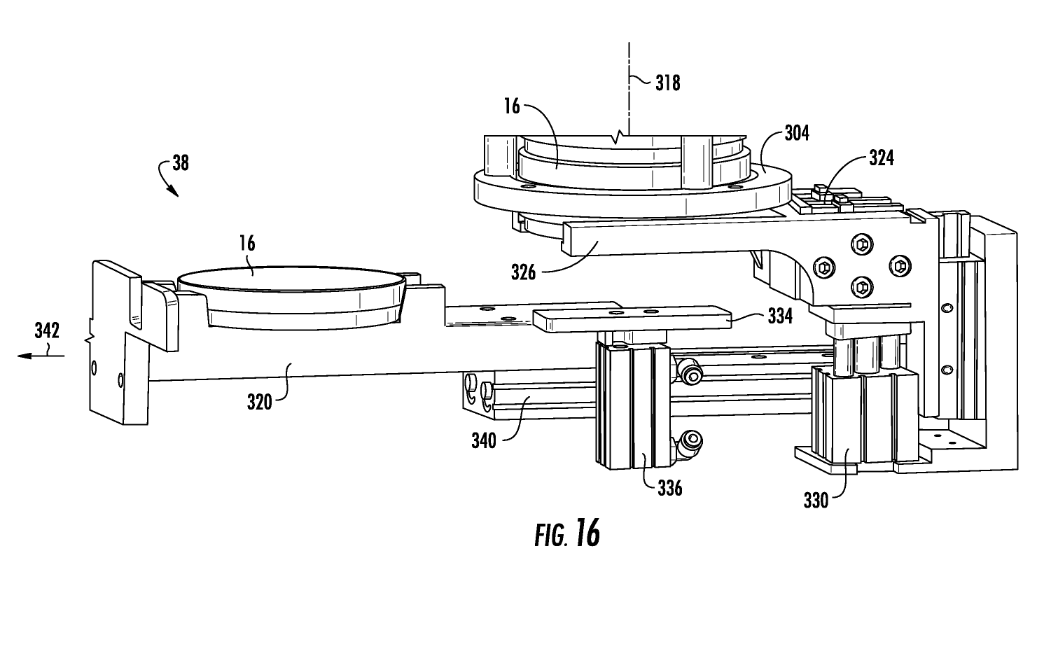

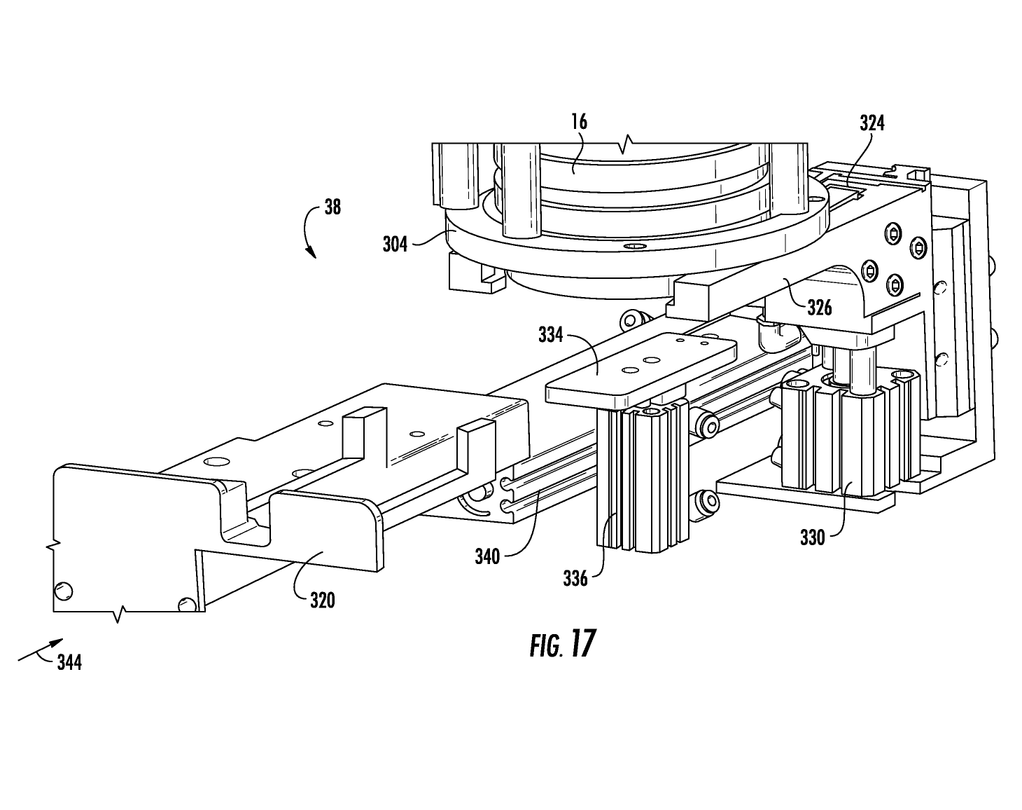

In the illustrative embodiment, the posts 306 and the curved plates 304, 312, 314 of the elongated body 302 define a passageway 316 centered about a longitudinal axis 318 extending from the distal end 310 and into the housing 300 (see FIGS. 14-19). A set of dishes 16 may be stacked within the passageway 316 such that the longitudinal axis 318 passed approximately through the center of each of the dishes 16.

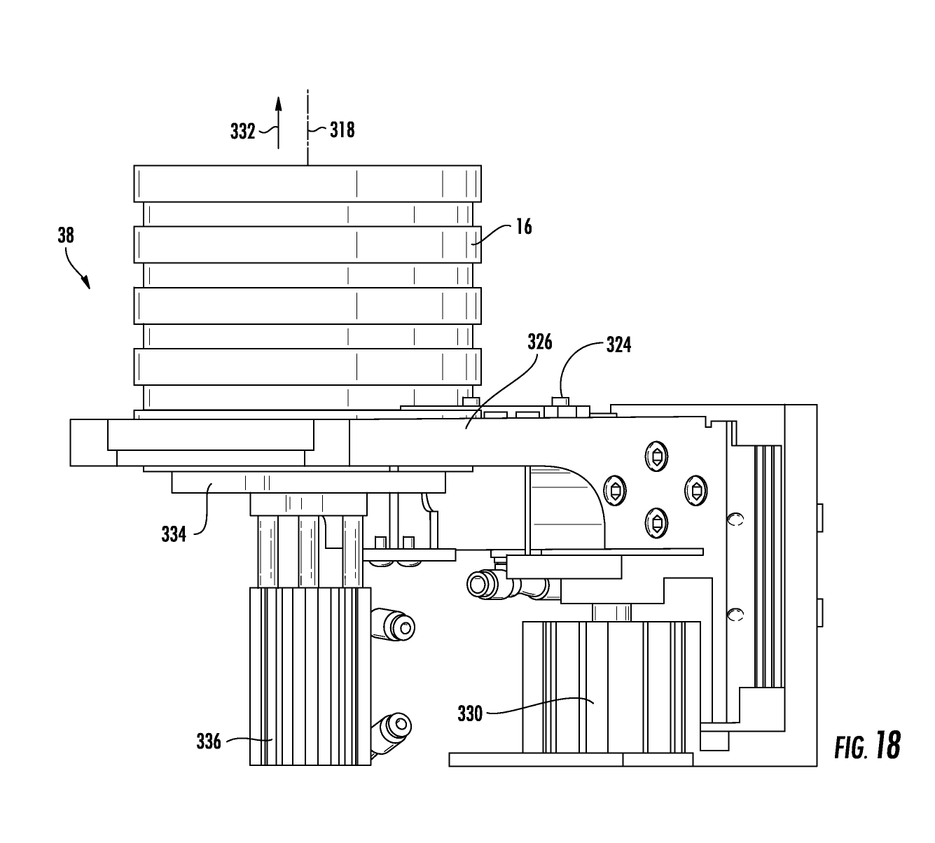

As shown in FIGS. 14-19, a plurality of pneumatic devices are included within the housing 300 of the dish dispensing system 38 and configured to move various components of the dish dispensing system 38 in order to retrieve a dish 16 from the stack of dishes 16 and extend the dish 16 away from the housing 300 so that the corresponding robotic arm 24 can retrieve the dish 16 for use in the system 10. For example, as shown in FIG. 13, in operation, a pneumatic device 340 (see FIG. 16) is configured to move a plate extender 320 holding a dish 16 from within the housing 300 to a position outside the housing 300 through a passageway 322 defined in the housing 300. It should be appreciated that one or more components of the dish dispensing system 38 may be omitted from the FIGS. 14-19 to emphasize other components and/or for clarity.

Referring now to FIGS. 14-19, components of the dish dispensing system 38 inside the housing 300 are shown without the housing 300 at various stages of operation of the dish dispensing system 38. As shown in FIG. 14, in operation, a pneumatic device 324 is configured to operate a pair of grip arms 326 to secure and/or release a dish 16 of the stack of dishes 16. In the illustrative embodiment, the grip arms 326 are parallel to one another and each of the grip arms 326 has a section 328 with a negative contour (not shown) defined therein that corresponding with the positive contour of a dish 16 with a lid 62. In the illustrative embodiment, the lid 62 of the dish 16 is configured to rest on a ledge (not shown) of the negative counter defined in the dish 16. As such, the grip arms 326 can secure the dish 16 without crushing it.

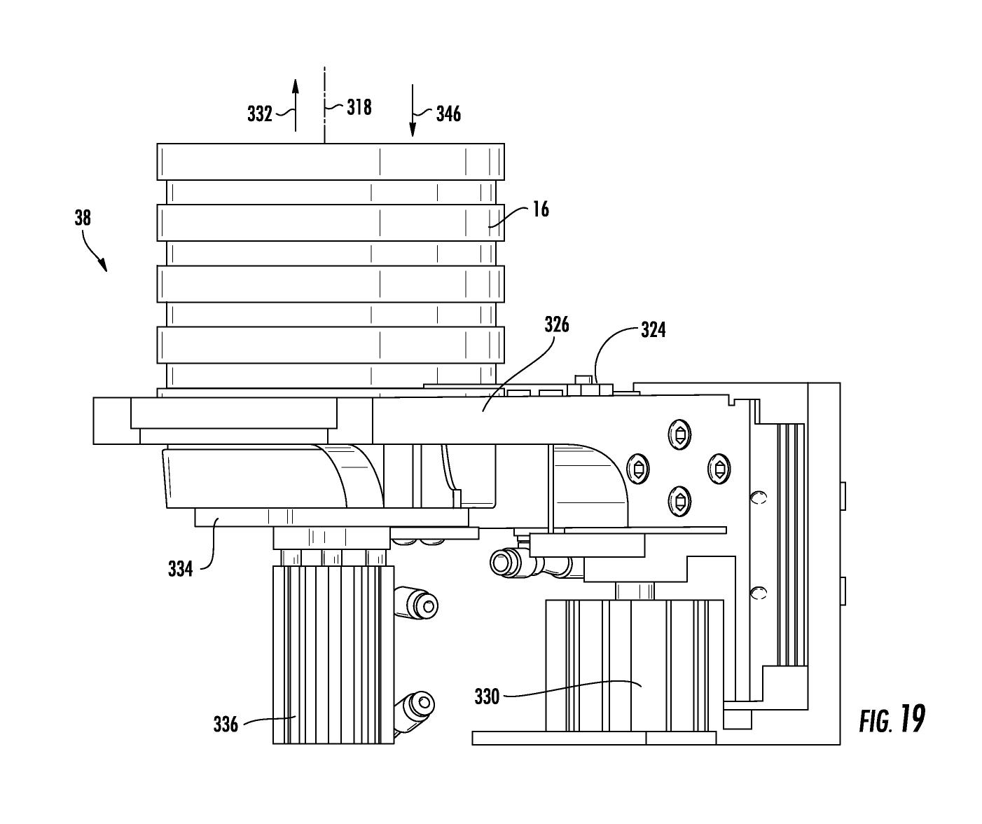

As shown in FIG. 15, in operation, the pneumatic device 324 closes the grip arms 326 to secure the dish 16 that is second from the bottom of the stack, and the pneumatic device 330 lifts the gripped dish 16 and the other dishes 16 stacked on the gripped dish 16 along the longitudinal axis 318 in the direction indicated by arrow 332. By doing so, the dish dispensing system 38 separates the bottom dish 16 from the stack of dishes 16. The bottom dish 16 is held in position by a dish riser 334 that is movable along the longitudinal axis 318 by the pneumatic device 336. As shown in FIG. 16, the pneumatic device 340 is operable to move the plate extender 320 and the bottom dish 16 supported by the dish riser 334 in a direction perpendicular to the longitudinal axis 318 as indicated by arrow 342. The plate extender 320 is configured to move the dish 16 through the passageway 322 to a position outside the housing 300 so that the robotic arm 24 can grasp the dish 16. As described below, the robotic arm 24 grasps the dish 16 from the plate extender 320 (see FIG. 17) and moves the dish 16 to the corresponding transfer station 30. The pneumatic device 340 retracts the plate extender 320 to a position in which it positioned between the stack of dishes 16 and the dish riser 334 by moving the plate extender in the direction indicated by arrow 344.

As shown in FIG. 18, in operation, after the dish 16 has been removed from the plate extender 320 and the plate extender 320 has been retracted, the pneumatic device 336 raises the dish riser 334. In particular, the pneumatic device 336 moves the dish riser 334 along the longitudinal axis 318 in the directed indicated by the arrow 332 until the dish riser 334 comes into contact with (or nearly into contact with) the bottom dish 16 of the stack of dishes 16 held by the grip arms 326. In such a way, the dish riser 334 is moved into a position such that it may support the weight of the stack of dishes 16. As shown in FIG. 19, the pneumatic devices 330, 324 operate in conjunction with one another to lower the stack of dishes 16 along the longitudinal axis 318 in the direction indicated by arrow 346. After the stack of dishes 16 is lowered, the pneumatic device 330 may open the grip arms 326 to release the bottom dish 16. It should be appreciated that the procedure described in reference to FIGS. 14-19 may be repeated each time the dish dispensing system 38 provides a dish 16 of cultivation media to the corresponding robotic arm 24.

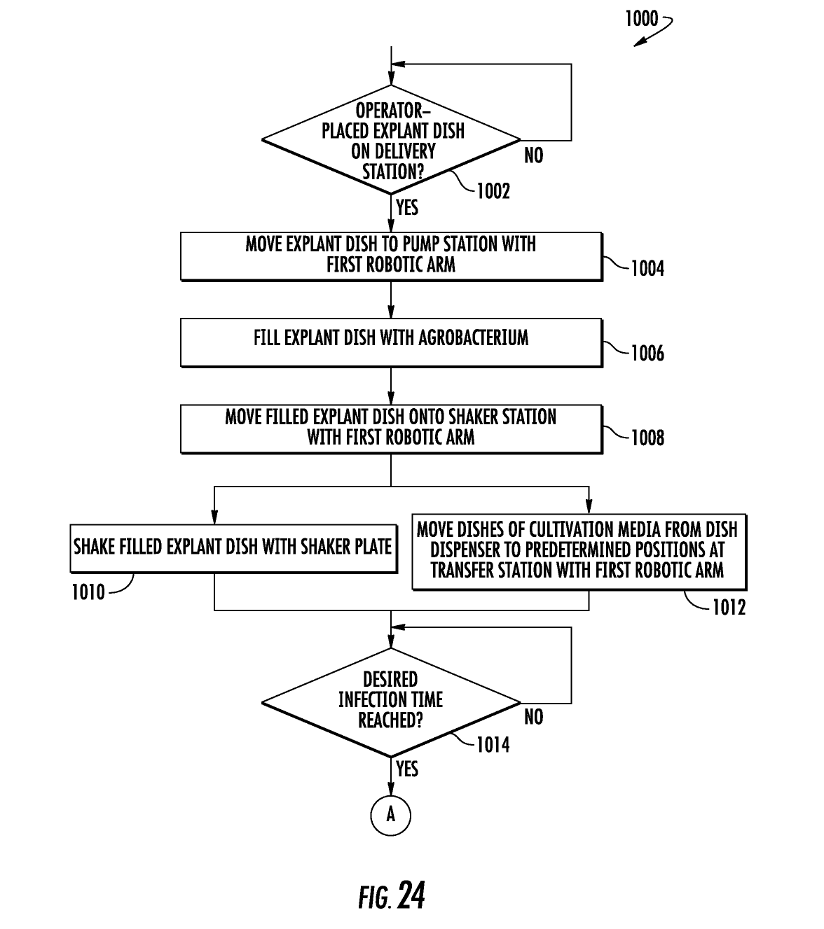

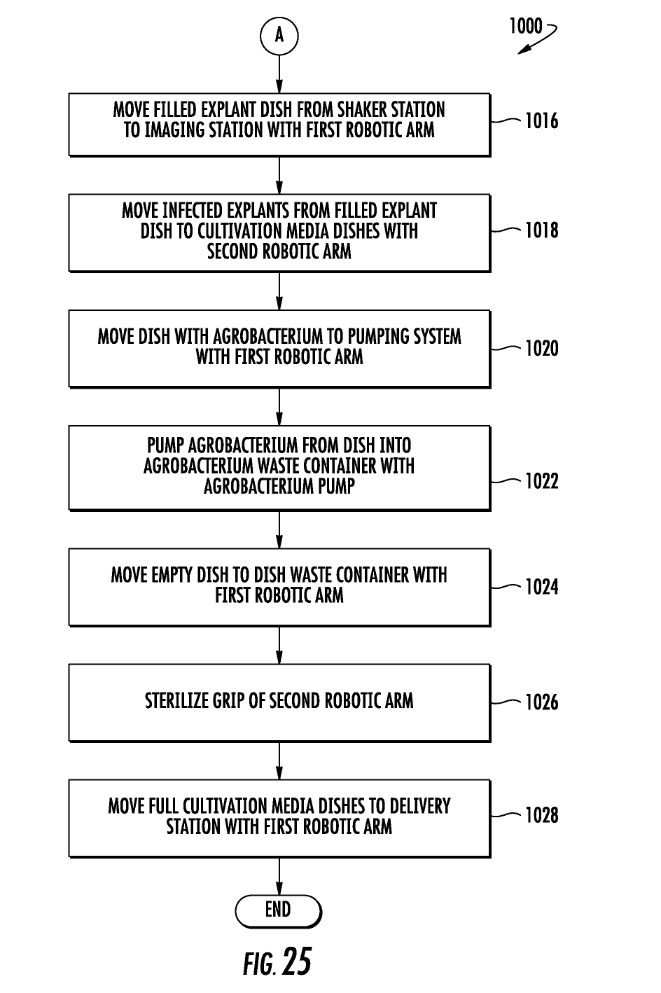

As described above, the system 10 includes a pair of delivery stations 28. In the illustrative embodiment, each of the delivery stations 28 is configured to serve multiple purposes. In particular, a user/operator of the system 10 may place a dish 16 of explants 12 on a deck 360 of each of the delivery stations 28 for use by the system 10. After operation of the system 10 has commenced, the controller 500 operates the corresponding robotic arm 24 to grasp the dish 16 of explants 12 and move the dish 16 to the pumping system 36 to be filled with an Agrobacterium solution as described below (see FIGS. 24-28). After the explants 12 have been infected with the Agrobacterium (i.e., at the corresponding shaker station 34) and placed onto the dishes 16 of cultivation media for growth, the robotic arm 24 moves the dishes 16 of cultivation media back to the corresponding delivery station 28 for access by the user/operator.

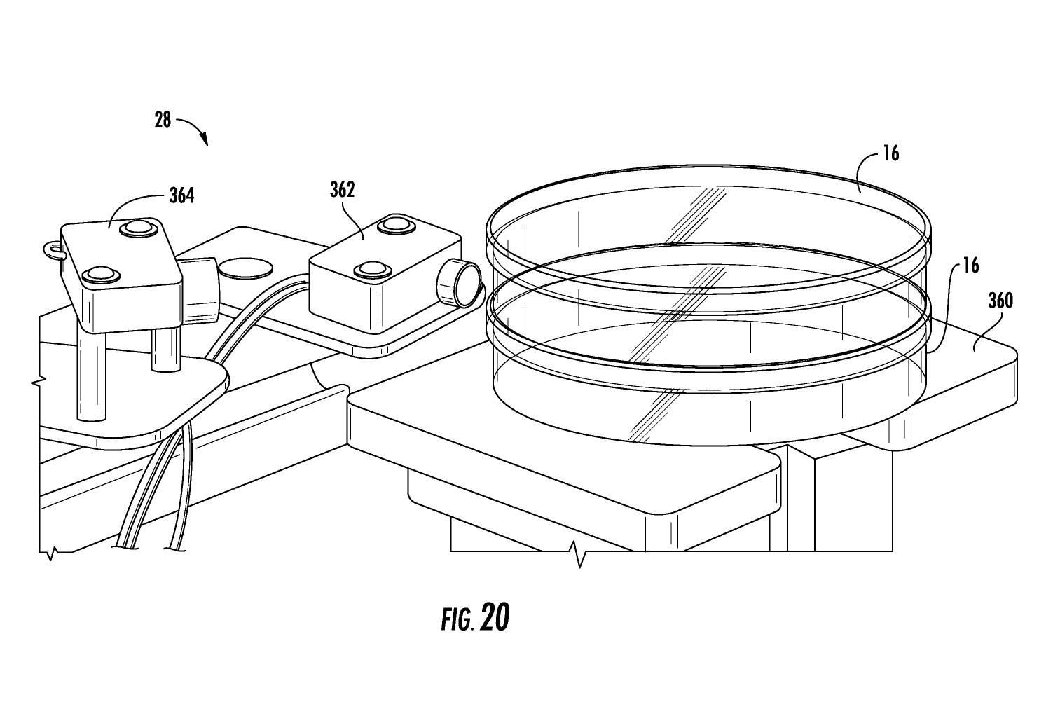

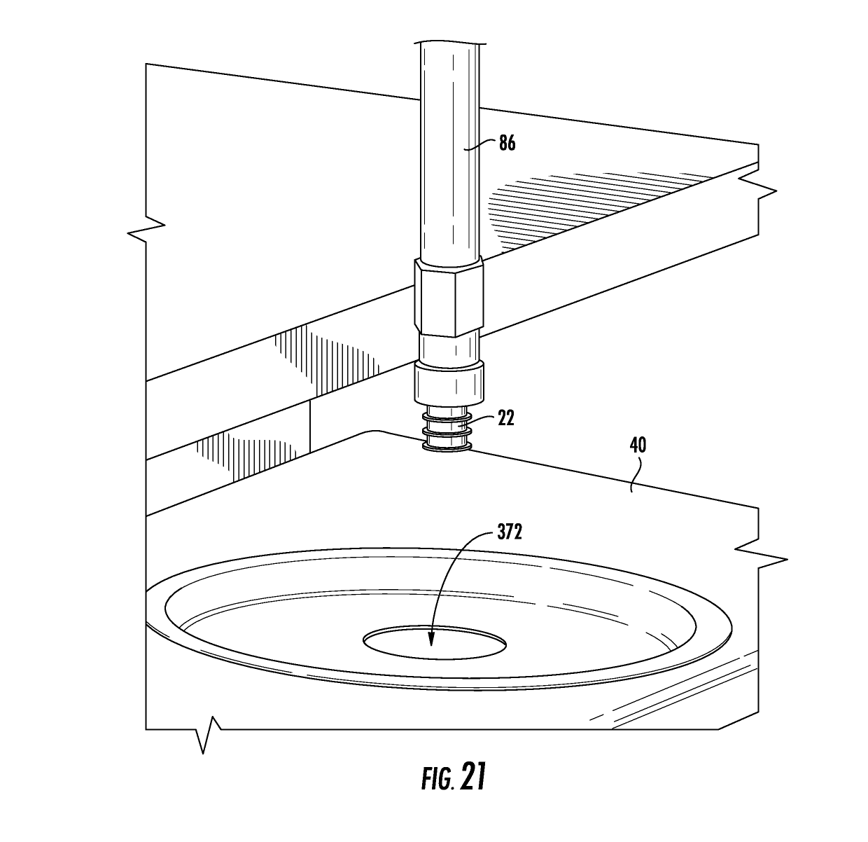

As shown in FIG. 20, the delivery station 28 includes two sensors 362, 364. In the illustrative embodiment, the sensors 362, 364 are embodied as type LV-NH32 adjustable spot sensors, commercially available from Keyence Corp., which are reflective type sensors in which the beam from a laser inside the sensor emits and is reflected back to the sensor if something is within the path of the beam, effectively sensing the presence of a dish 16. The sensor 362 is configured to sense the presence of a first dish 16 or bottom dish 16 positioned on deck 360, whereas the sensor 364 is configured to sense the presence of a second dish 16 stacked on top of the first dish 16, which is indicative of a stack of dishes 16. As such, the controller 500 may utilize the sensor data of the sensors 362, 364 to determine the state of the delivery station 28 (e.g., that no dishes 16 are present, that one dish 16 is present, or that multiple dishes 16 are present). Further, the state may be conveyed to the user by the controller 500 and/or used by the system 10 (e.g., to confirm when the explants 12 are available for pickup by the robotic arm 24). Although the illustrative delivery station 28 includes two adjustable spot sensors, it should be appreciated that other embodiments may use a different number and/or type of sensors. For example, in some embodiments, the sensors 362, 364 may be embodied as optical sensors, light sensors, pressure sensors, image sensors, motion sensors, inertial sensors, piezoelectric sensors, and/or any other type of sensors suitable for performing the functions described herein.

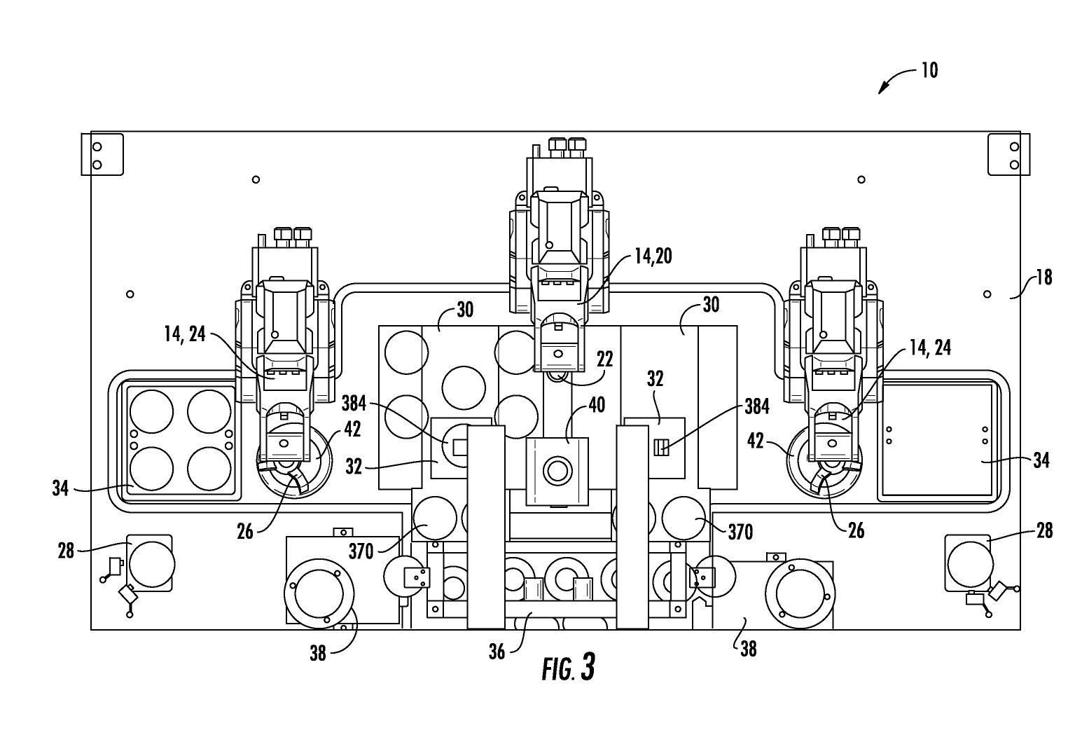

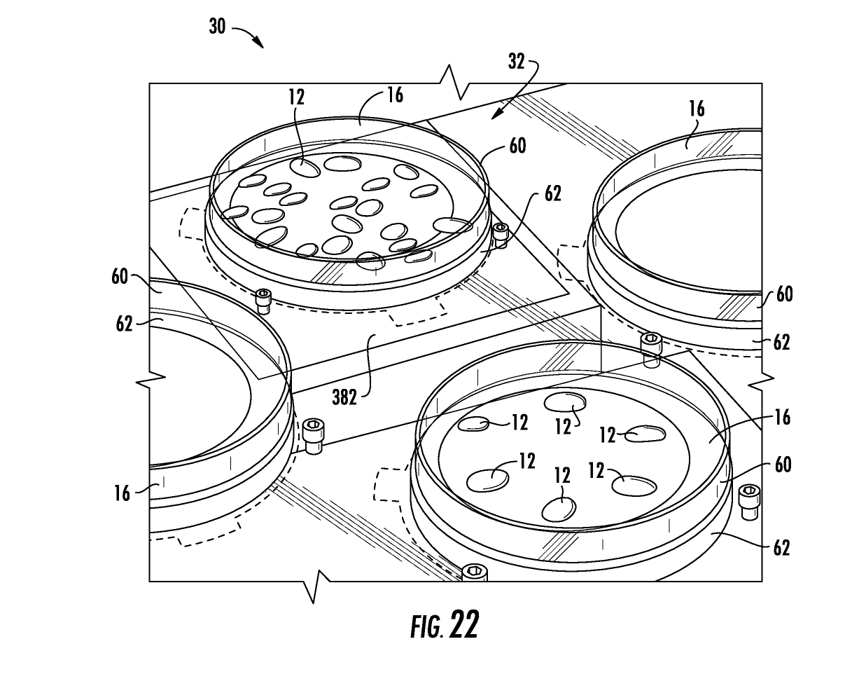

Referring now to FIG. 21, the system 10 includes a sterilization device 40 that is configured to sterilize the suction grip 22 of the robotic arm 20. To do so, the controller 500 operates the robotic arm 20 to insert the suction grip 22 into a container 370 (see FIGS. 2-3) filled with ethanol or another suitable sterilizing solution. In the illustrative embodiment, the solution contains 70% alcohol. The robotic arm 20 may be operated to move the grip 22 up and down and side to side within the ethanol for some period of time before advancing the grip 22 into an opening 372 of the sterilization device 40, as shown in FIG. 21. In the illustrative embodiment, the sterilization device 40 is a dry glass bead sterilizer such as, for example, an InoTech BioScience Steri 250. The robotic arm 20 may again be operated to move the grip 22 up and down within the sterilizer 40 for a few seconds in the illustrative embodiment. The robotic arm 20 may then withdraw the grip 22 from the sterilizer 40 so that the grip 22 is permitted to cool. Due to the heat generated by the sterilizer 40, the bellows of the grip 22 may become stuck together such that performance of the grip 22 may be impaired. In those circumstances, the robotic arm 20 may perform a procedure to separate the bellow (e.g., by suctioning a sterile surface and stretching the bellows).

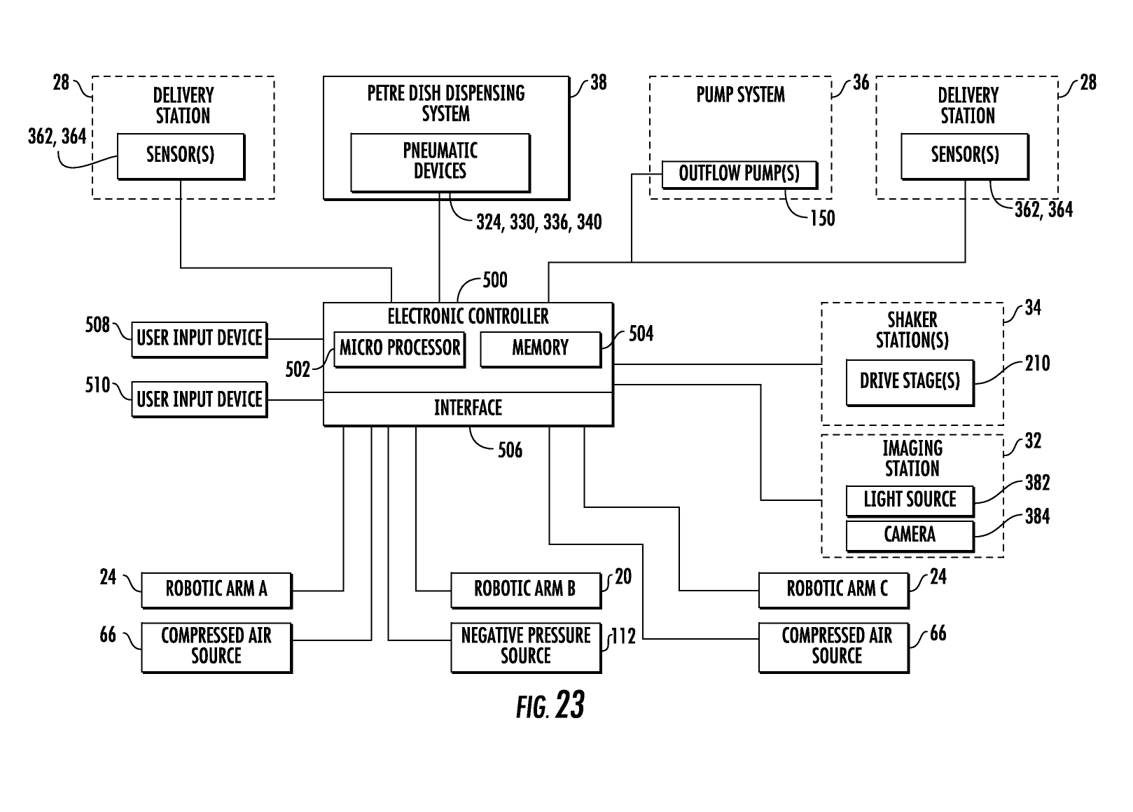

As described above, the transfer stations 30 are used to transfer explants 12 infected with an Agrobacterium solution to dishes 16 including cultivation media (e.g., agar). Referring now to FIG. 22, a portion of one of the transfer stations 30 is shown. As described above, the transfer station 30 includes an imaging station 32 that is configured to capture images of a dish 16 of explants 12, which are analyzed by the controller 500 to determine the locations of the explants 12 on the dish 16. It should be appreciated that the robotic arm 20 can grasp a particular explant 12 based on its determined location on the dish 16. In the illustrative embodiment, the transfer station 30 includes a transparent deck 380 on which a plurality of dishes 16 may be placed. For example, the transparent deck 380 may be composed of Plexiglas, acrylic, glass, and/or another suitable transparent material. In other embodiments, the deck 380 may be opaque or semi-transparent in one or more portions of the deck 380 (e.g., outside the imaging station 32).

The imaging station 32 includes a light source 382 positioned below the transparent deck 380 and configured to illuminate the portion of the transparent deck 380 corresponding with the imaging station 32 so as to illuminate explants 12 within a dish 16 placed on the imaging station 32. The light source 382 is illustratively embodied as a red light-emitting diode (LED). It should be appreciated that in other embodiments other colored LEDs may be used. In still other embodiments, other lighting sources may be used.

The system 10 includes a camera 384, which is mounted above the imaging station 32 from a riser (see FIG. 3). The camera 384 is operable to capture images of the contents of the dish 16 at the imaging station 32. In the illustrative embodiment, the camera 384 is configured to capture black and white images; however, the camera 384 may be configured to capture colored, grayscale, and/or other types of images in other embodiments. It should be appreciated that by properly setting the aperture of the camera 384, all or nearly all traces of transparent objects (e.g., a dish 16) in a captured image may be eliminated. Further, with a black and white camera, red light emitted from the light source 382 appears bright white in a captured image and solid objects (e.g., seed explants 12) appear black. The camera 384 is electrically coupled to an electronic controller 500 (see FIG. 23). As described in greater detail below, the images may be sent to the controller 500 to determine the relative locations and orientations of the explants 12 in the dish 16 such that the system 10 can direct the robotic arm 20 to the explants 12 for retrieval.

Referring now to FIG. 23, the system 10 includes an electronic controller 500. The controller 500 is, in essence, the master computer responsible for interpreting electrical signals sent by sensors associated with the system 10 and for activating or energizing electronically-controlled components associated with the system 10. For example, the electronic controller 500 is configured to control the operation of the sensors 362, 364, pneumatic devices 324, 330, 336, 340, pumps 150, drive stages 210, camera 384, and so forth. While the electronic controller 500 is shown as a single unit in FIG. 23, the controller 500 may include a number of individual controllers for the various components as well as a central computer that sends and receives signals from the various individual controllers. The electronic controller 500 also determines when various operations of the system 10 should be performed. As will be described in more detail below, the electronic controller 500 is operable to control the components of the system 10 such that the system 10 selects and processes soybean explants 12 for use in transgenic protocols.

To do so, the electronic controller 500 includes a number of electronic components commonly associated with electronic units utilized in the control of electromechanical systems. For example, the electronic controller 500 may include, amongst other components customarily included in such devices, a processor such as a microprocessor 502 and a memory device 504 such as a programmable read-only memory device (“PROM”) including erasable PROM's (EPROM's or EEPROM's). The memory device 504 is provided to store, amongst other things, instructions in the form of, for example, a software routine (or routines) which, when executed by the microprocessor 502, allows the electronic controller 500 to control operation of the system 10.

The electronic controller 500 also includes an analog interface circuit 506. The analog interface circuit 506 converts the output signals from the various components into signals that are suitable for presentation to an input of the microprocessor 502. In particular, the analog interface circuit 506, by use of an analog-to-digital (A/D) converter (not shown) or the like, converts the analog signals generated by the sensors into digital signals for use by the microprocessor 502. It should be appreciated that the A/D converter may be embodied as a discrete device or number of devices, or may be integrated into the microprocessor 502. It should also be appreciated that if any one or more of the sensors associated with the system 10 generate a digital output signal, the analog interface circuit 506 may be bypassed.

Similarly, the analog interface circuit 506 converts signals from the microprocessor 502 into output signals which are suitable for presentation to the electrically-controlled components associated with the system 10 (e.g., the robotic arms 14). In particular, the analog interface circuit 506, by use of a digital-to-analog (D/A) converter (not shown) or the like, converts the digital signals generated by the microprocessor 502 into analog signals for use by the electronically-controlled components associated with the system 10. It should be appreciated that, similar to the A/D converter described above, the D/A converter may be embodied as a discrete device or number of devices, or may be integrated into the microprocessor 502. It should also be appreciated that if any one or more of the electronically-controlled components associated with the system 10 operate on a digital input signal, the analog interface circuit 506 may be bypassed.

Thus, the electronic controller 500 may operate to control the operation of the system 10. In particular, the electronic controller 500 executes a routine including, amongst other things, a control scheme in which the electronic controller 500 monitors the outputs of the sensors associated with the system 10 and controls the inputs to the electronically-controlled components of the system 10. To do so, the electronic controller 500 performs numerous calculations, either continuously or intermittently, including looking up values in preprogrammed tables, in order to execute algorithms to perform such functions as energizing the robotic arms 14, energizing the pumps 150, varying the light intensity of the light source 382 to improve image contrast, and so on. In some embodiments, the controller 500 may also include a user input device 508 to receive input from the user of the system 10 and/or a user output device 510 to provide output to the user. The user input device 508 may be embodied as any integrated or peripheral device such as a keyboard, mouse, touchscreen, and/or other input devices configured to perform the functions described herein. Similarly, the user output device 510 may be embodied as any integrated or peripheral device such as a display, speaker, and/or other output devices configured to perform the functions described herein.

Referring now to FIGS. 24-25, an illustrative operating procedure 1000 for automated explant preparation is shown. It will be appreciated that prior to commencement of the procedure 1000, the controller 500 may calibrate the system 10, provide messages to the user, retrieve user input, initialize safety mechanisms (e.g., a light curtain), and perform other setup functions. For example, if not done already, the controller 500 may calibrate the system 10 using any suitable protocol to map or otherwise correlate the coordinate system of the robotic arms 20 to the coordinate systems of the camera 384 such that locations of objects captured in images may be translated to a location of that object relative to the arms 20. Further, the controller 500 may calibrate the system 10 to correlate the coordinate system of the robotic arms 20, 24 with various predefined locations of the system 10 (e.g., specific points on the transfer station 30, shaker station 34, delivery station 28, pumping system 36, dish dispensing system 38, etc.) to ensure that the robotic arms 20, 24 retrieve and drop the relevant explants 12 and/or dishes 16 in the appropriate locations. Additionally, in some embodiments, the controller 500 may provide setup instructions to the user on a display or other user output device 510 (e.g., to place a dish 16 of explants 12 on the delivery stations 28) and/or retrieve input from the user via a user input device 508 (e.g., to pause the system 10).

In block 1002, the system 10 determines whether the operator has placed a dish 16 of explants 12 on the delivery station(s) 28. As discussed above, in some embodiments, the system 10 makes such a determination based on sensor data generated by the sensors 362, 364. For clarity of the description, the procedure 1000 is described herein with respect to one “side” of the system 10 or the table 18 (e.g., one robotic arm 24); however, it should be appreciated that the procedure 1000 may be performed by both sides of the system 10 in parallel. If the explant dish 16 has been placed on the delivery station 28, the procedure 1000 advances to block 1004 in which the controller 500 operates the robotic arm 24 to grasp the explant dish 16 and move the explant dish 16 from the delivery station 28 to the pumping system 36. In particular, as described above, the robotic arm 24 moves the explant dish 16 to the fluid delivery station 160.

The procedure 1000 advances to block 1006 in which the controller 500 operates one of the pumps 150 to fill the explant dish 16 with an Agrobacterium tumefaciens solution. In some embodiments, it should be appreciated that the user may desire to utilize multiple different types of solutions in a particular experiment. In such embodiments, the pumps 150 of the pumping system 36 may be configured to extract different solutions, and the controller 500 may control the pumping system 36 to ensure the appropriate solution is delivered to the dish 16 at a given time. In block 1008, the controller 500 operates the robotic arm 24 to move the filled explant dish 16 onto a predefined location of the corresponding shaker station 34. In the illustrative embodiment, the shaker station 34 has four predefined locations on the shaker plate 202 at which the dishes 16 may be placed such that four explant dishes 16 may be processed (i.e., agitated) by the shaker station 34. As described above, the robotic arm 24 may be calibrated during initialization to store data associated with those locations (i.e., to “remember” the locations).

In block 1010, the shaker station 34 is configured to agitate/shake the dish(es) 16 of explants 12 in order to infect the explants 12 with the Agrobacterium tumefaciens solution. In some embodiments, the controller 500 may utilize a timer to track a processing time of a particular dish 16 by the shaker station 34. While the shaker station 34 is processing the dish(es) 16 of explants 12, the controller 500 operates the robotic arm 24 to move the dishes 16 of cultivation media (e.g., agar) from the dish dispensing system 38 to predetermined positions at the transfer station 30. In the illustrative embodiment, the controller 500 instructs the robotic arm 24 to move five cultivation media dishes 16 to five separate predetermined/calibrated positions on the transfer station 30 as shown in FIG. 3.

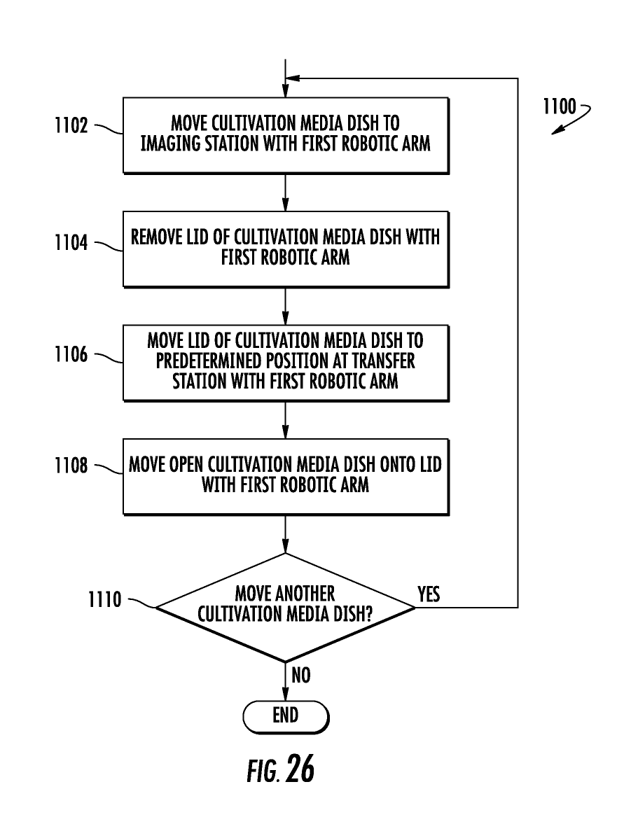

In the illustrative embodiment, a procedure 1100 may be used to move the cultivation media dishes 16 to the transfer station 30 as shown in FIG. 26. The procedure 1100 begins with block 1102 in which the controller 500 operates the robotic arm 24 to grasp a dish 16 of cultivation media from the dish dispensing system 38 (i.e., from the plate extender 320) and to move the dish 16 to the imaging station 32. The procedure 1100 advances to block 1104 in which the controller 500 operates the robotic arm 24 to grasp the lid 62 of the cultivation media dish 16 and remove the lid 62 from the dish 16. In block 1106, the controller 500 operates the robotic arm 24 to move the lid 62 of the dish 16 to a predetermined position at the transfer station 30 (e.g., one of the five predetermined positions described above). In block 1108, the controller 500 operates the robotic arm 24 to grasp and move the open cultivation media dish 16 (i.e., the base 60 of the dish 16) at the imaging station 32 to the position on the transfer station 30 at which the corresponding lid 62 is located. In other words, the robotic arm 24 places the base 60 of the dish 16 on top of the corresponding lid 62, as shown in FIG. 22.

The procedure 1100 advances to block 1110 in which the controller 500 determines whether to move another cultivation media dish 16. As described above, in the illustrative embodiment, the controller 500 is programmed to move five cultivation media dishes 16 from the dish dispensing system 38 to the predefined positions on the transfer station 30. Accordingly, in the illustrative embodiment, the controller 500 determines whether it has already moved five cultivation media dishes 16 to the transfer station 30. If so, the procedure 1110 terminates. Otherwise, the procedure 1110 returns to block 1102 in which the controller 500 instructs the robotic arm 24 to grasp another cultivation media dish 16. Although the illustrative embodiments describes the use of five cultivation media dishes 16, in other embodiments, the system 10 may utilize any suitable number of cultivation media dishes 16 consistent with the techniques described herein.

Returning to FIG. 24, after the controller 500 moves the appropriate number of cultivation media dishes 16 to the transfer station 30. As shown in FIG. 26, the procedure 1000 advances to block 1014 in which the controller 500 determines whether the explant dish(es) 16 have been sufficiently processed by the shaker station 34 to fully infect the explants 12 with the Agrobacterium tumefaciens solution. For example, in the illustrative embodiment, the controller 500 utilizes a timer to confirm that a particular explant dish 16 has been agitated by the shaker station 34 for a predetermined threshold infection time (e.g., 30 minutes). However, in other embodiments, it should be appreciated that the system 10 may utilize any other suitable condition(s) and/or techniques to determine whether the explants 12 have been infected.

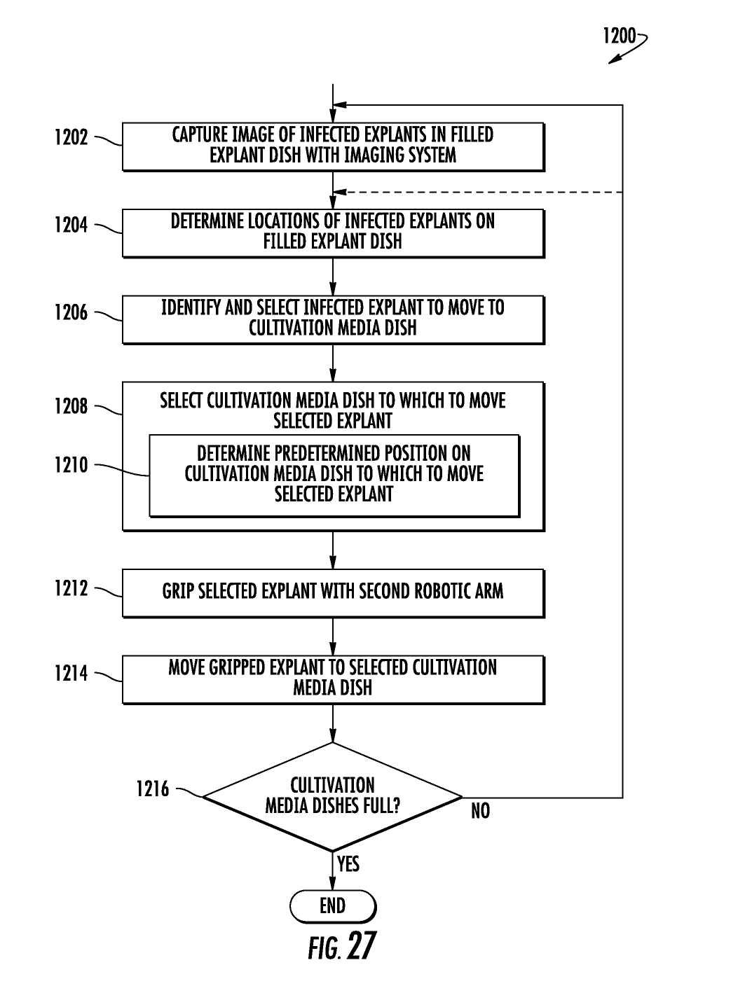

If the desired infection time has been reached (or other infection condition satisfied), the procedure 1000 advances to block 1016 of FIG. 25 in which the controller 500 selects an explant dish 16 from the shaker station 34 (e.g., the explant dish 16 for which the infection timer expired) and operates the robotic arm 24 to grasp and move the explant dish 16 to the imaging station 32. The procedure 1000 advances to block 1018 in which the controller 500 operates the robotic arm 20 to move the infected explants 12 from the explant dish 16 at the imaging station 32 to predetermined positions on the cultivation media dishes 16. To do so, an illustrative procedure 1200, as shown in FIG. 27, may be used.

Referring now to FIG. 27, the procedure 1200 begins with block 1202 in which the controller 500 operates the camera 384 to capture an image of the infected explants 12 in the dish 16 at the imaging station 32. One such image 600 is showed in FIG. 30. As shown in FIG. 30, the explants 12 may be positioned in arbitrary locations and orientations relative to one another within the dish 16. In block 1204, the controller 500 may process the captured image 600 to identify the locations of the infected explants 12 on the dish 16. In some embodiments, the controller 500 is configured to determine the locations of all of the identifiable explants 12, whereas in other embodiments, the controller is configured to identify only a single explant 12.

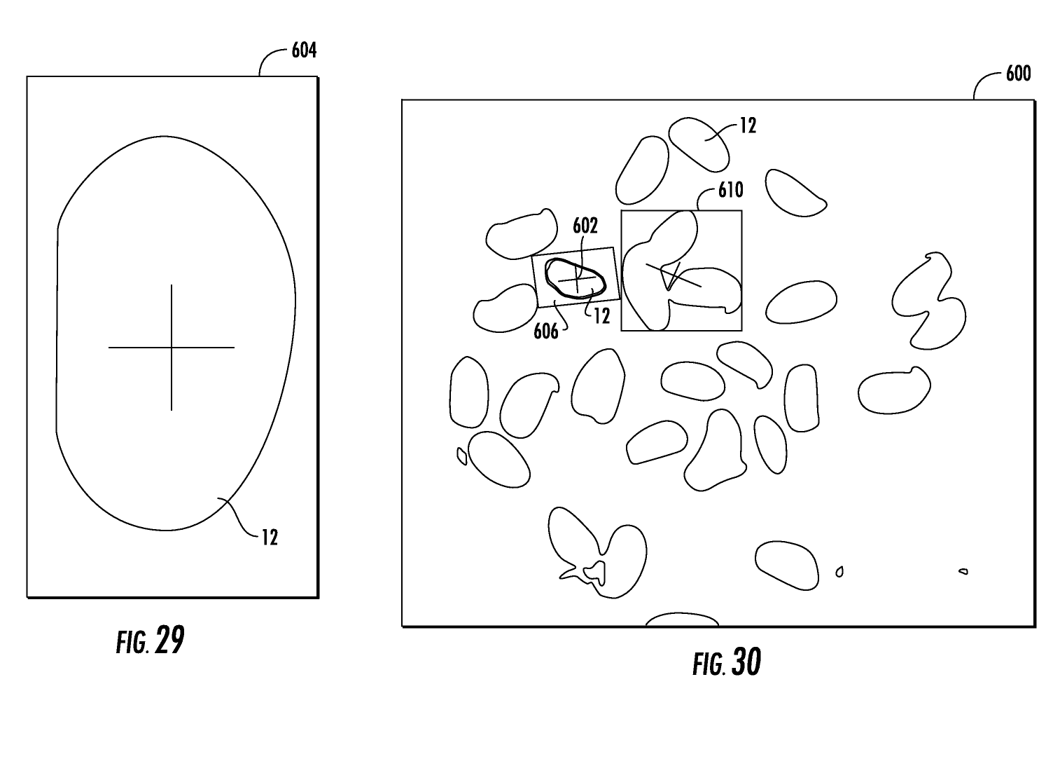

It should be appreciated that the controller 500 may utilize any suitable image processing technique to determine the locations of the explants. For example, in the illustrative embodiment, the controller 500 converts the image to a binary image (i.e., black and white) and utilizes a geometric object-identifying function of the software package included with the Epson model C3 six-axis articulated arms. In particular, a reference image 604 of the explant 12 (see FIG. 29) loaded by the user and stored in the memory device 504 of the controller 500 is compared to the captured image 600 of the explants 12 to identify a match 606. The geometric object-identifying function employs an algorithmic approach that identifies matches to a reference image (i.e., an object model) by using edge-based geometric features. Further, the geometric object-identifying function includes various parameters such as a reference image to be used for comparison to another image, an acceptance or tolerance level required for the match 606, minimum or maximum object size for the match 606, and/or other suitable parameters.

If the controller 500 is unable to locate an individual explant 12 separated from other explants 12 but locates a group 610 of explants 12 (e.g., overlapping explants 12), the controller 500 executes a protocol to separate the group 610 of overlapping explants 12. For example, in the illustrative embodiment, the controller 500 may identify a geometric center of the group 610 using a suitable imaging algorithm (e.g., by detecting a center of mass of the group) and instructs the robotic arm 20 to insert the suction grip 22 into the bin 198 of the dish 16 (e.g., into the Agrobacterium solution) and to stir or agitate the group 610 of explants 12 in order to disperse them. In other embodiments, the controller 500 may instruct the robotic arm 20 to grasp and drop one of the explants 12 in the group 610 in order to separate them. In yet other embodiments, the controller 500 may move the suction grip 22 to a location of the group 610 in the bin 198 and operate the negative pressure source 112 in reverse (if possible with the particular system 10) to expel compressed air into the bin 198 to separate the explants 12.

It should be appreciated that the system 10 may utilize any other suitable mechanism for separating the group 610 of explants 12 in other embodiments. Further, the controller 500 may utilize any suitable image processing algorithms and techniques to identify the locations of the explants 12 in the dish 16. For example, the controller 500 may utilize feature detection algorithms, techniques, and filters such as Speeded Up Robust Features (SURF), Scale-Invariant Feature Transform (SIFT), Multi-Scale Oriented Patches (MOPS), Canny, image gradient operators, and Sobel filters to identify features (e.g., interest points such as corners, edges, blobs, etc.) of the image 600 and the explant reference image 604. In some embodiments, the controller 500 may utilize feature matching algorithms such as the Random Sample Consensus (RANSAC) algorithm to determine whether any features identified in the image 600 and the explant reference image 604 correspond with one another and, if so, the corresponding locations of those features. Additionally or alternatively, the controller 500 may utilize image segmentation algorithms (e.g., pyramid segmentation, watershed algorithms, etc.) for identifying objects in an image. It will be appreciated that, depending on the particular embodiment, the controller 500 may utilize any one or more of the algorithms described above during the analyses of captured images.

After the controller 500 determines the location(s) of the explant(s) 16, the procedure 1200 advances to block 1206. In block 1206, the controller 500 identifies and selects (e.g., arbitrarily or algorithmically) an infected explant 12 to move to a cultivation media dish 16 on the transfer station 30 as described above. In block 1208, the controller 500 selects a cultivation media dish 16 to which to move the selected explant 12. More particularly, in block 1210, the controller 500 determines a predetermined location on the cultivation media dish 16 at which to place the selected explant 12.

In the illustrative embodiment, the original dish 16 of explants 12 provided by the user/operator of the system 10 (see block 1002 of FIG. 24) holds approximately thirty seed explants, and the controller 500 is configured to place six explants 12 on each of the five cultivation media dishes 16 in predetermined locations. For example, the controller 500 may be configured to place the explants 12 on a cultivation media dish 16 in a circle at equal distances from one another (e.g., approximately 60 degrees apart). Accordingly, in the illustrative embodiment, the controller 500 selects the cultivation media dish 16 and the location at which to place the explant 12 on that cultivation media dish 16 based on the previous locations at which explants 12 have been placed. In the illustrative embodiment, the controller 500 stores the previous locations (i.e., locations at which explants 12 are currently placed) in the memory 504 in order to prevent multiple explants 12 from being placed at the same location. However, in other embodiments, the system 10 may utilize, for example, a camera and image processing technique to make such a determination.

The procedure 1200 advances to block 1212 in which the controller 500 operates the robotic arm 20 to grip the selected explant 12 from the dish 16 at the imaging station 32. It should be appreciated that to grasp the explant 12 from the dish 16, the grip assembly 80 is positioned above a grip location/point of the explant 12 (e.g., the center of the explant 12) such that the hollow passageway 106 of the grip assembly 80 is approximately collinear with the grip location. The grip assembly 80 is then advanced downward toward the explant 12 until the suction grip 22 is in full contact with the outer surface of the explant 12. As described above, the suspension mechanism 86 operates to prevent the explant 12 from being crushed while ensuring that the grip 22 is in full contact with the explant 12's surface to provide limited loss of suction. The negative pressure source 112 may then be activated to secure the explant to the grip 22.

In block 1214, the controller 500 operates the robotic arm 20 to move the gripped explant 12 to the selected cultivation media dish 16 and the determined position on the dish 16. In block 1216, the controller 500 determines whether each of the cultivation media dishes 16 is full. If so, the procedure 1200 terminates. Otherwise, the procedure 1200 returns to block 1202 in which the controller 500 instructs the camera 384 to capture another image of the dish 16 at the imaging station 32. In some embodiments, the procedure 1200 may utilize the original image 600 captured by the camera 384 (denoted by the dashed arrow in FIG. 27). As described above, in the illustrative embodiment, a cultivation media dish 16 is considered to be “full” if it has six explants 12 on the dish 16. In other embodiments, the controller 500 may, additionally or alternatively, use other criteria for making such a determination. For example, in some embodiments, the controller 500 may determine whether there are any explants 12 remaining on the dish 16 at the imaging station 32; if not, the procedure 1200 may terminate.

In some embodiments, the system 10 may be configured to space a predetermined number (n) of the explants 12 apart evenly on each cultivation media dish 16 such that the explants 12 are spaced approximately 360/n degrees apart from one another on the cultivation media dish 16. Further, in some embodiments, the predetermined number (n) of explants 12 to place on a particular cultivation media dish 16 may be selected by an operator of the system 10. For example, in embodiments in which the operator selects, or the system 10 otherwise determines, to place six explants 12 on each cultivation media dish 16, those six explants 12 would be spaced approximately 60 degrees (360/60=60) apart from one another on the corresponding cultivation media dish 16. In an embodiment in which the system 10 determines to place four explants 12 on each cultivation media dish 16, those four explants 12 would be spaced approximately 90 degrees (360/4=90) apart from one another on the corresponding cultivation media dish 16. In such embodiments, the cultivation media dish 16 may be considered to be “full” if all n explants 12 are placed (e.g., evenly) on the cultivation media dish 16.

Returning to FIG. 25, after the infected explants 12 have been moved to the cultivation media dishes 16, the procedure 1000 advances to block 1020. In block 1020, the controller 500 instructs the robotic arm 24 to grasp and move the dish 16 at the imaging station 32 from which the infected explants 12 were moved to the pumping system 36 or, more particularly, to the fluid extraction station 162. As described above, the robotic arm 24 moves the dish 16 into a position such that the distal end 196 of the extraction tube 182 is positioned within the bin 198 of the dish 16. In block 1022, the controller 500 operates the appropriate pump 150 to extract/pump the Agrobacterium solution from the dish 16 into the corresponding solution container 152 (for used solution). As described above, the controller 500 may contemporaneously operate the robotic arm 24 to tilt the dish 16 toward the extraction tube 182 during extraction in order to ensure that all, or a majority, of the Agrobacterium is removed from the dish 16.

The procedure 1000 advances to block 1024 in which the controller 500 operates the robotic arm 24 to move the empty dish 16 to the appropriate dish waste container 42. The robotic arm 24 releases its grip to drop the dish 16 into the waste container 42. It should be appreciated that by removing the Agrobacterium solution from the dish 16 prior to disposal of the dish 16, the risk of the Agrobacterium spilling or splashing during disposal is reduced or minimized. In block 1026, the controller 500 operates the robotic arm 20 to sterilize the suction grip 22. To do so, the controller 500 may use a procedure similar to the procedure described above in reference to FIG. 21.

In block 1028, the controller 500 operates the robotic arm 24 to move the “full” cultivation media dishes 16 with the infected explants 12 to the delivery station 28. As described above, the cultivation media dishes 16 may be stacked on the delivery station 28 for retrieval by the user/operator of the system 10. Further, the controller 500 may notify the user/operator that the cultivation media dishes 16 are available for pickup via the user output device 510 upon completion.

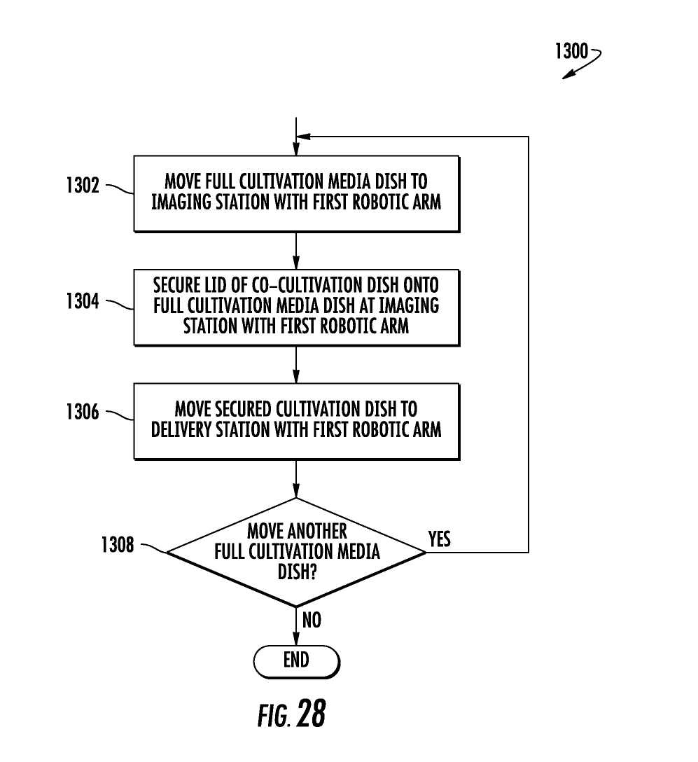

In the illustrative embodiment, a procedure 1300 may be used to move the full cultivation media dishes 16 to the delivery station 28 as shown in FIG. 28. The procedure 1300 of begins with block 1302 in which the controller 500 operates the robotic arm 24 to select (arbitrarily or algorithmically) and move one of the full cultivation media dishes 16 with the infected explants 12 to the imaging station 32. As described above, in the illustrative embodiment, the cultivation media dishes 16 were originally placed on the transfer station 30 such that the base 60 of each dish 16 was placed on top of its lid 62. Accordingly, in the illustrative embodiment, the controller 500 more specifically operates the robotic arm 24 to grasp and move the base 60 of a cultivation media dish 16 to the imaging station 32.

In block 1304, the robotic arm 24 secures the lid 62 to the cultivation media dish base 60 moved to the imaging station 32. That is, the controller 500 operates the robotic arm 24 to grasp the lid 62 of the selected cultivation media dish 16 from the transfer station 30 and moves the lid 62 onto the base 60 of the cultivation media dish 16 at the imaging station 32. In block 1306, the controller 500 operates the robotic arm 24 to move the secured cultivation media dish 16 with infected explants 12 to the delivery station 28. As discussed above, if another cultivation media dish 16 is already placed on the delivery station 28, the robotic arm 24 stacks the dishes 16.

The procedure 1300 advances to block 1308 in which the controller 500 determines whether to move another full cultivation media dish 16. In other words, the controller 500 determines whether any cultivation media dishes 16 remain on the transfer station 30. If not, the procedure 1300 terminates. Otherwise, the procedure 1300 returns to block 1302 to repeat the procedure 1300 and move another full cultivation media dish 16 to the delivery station 28. In the illustrative embodiment, because the system 10 moves infected explants 12 to five cultivation dishes 16 on the transfer station 30, it should be appreciated that the system 10 stacks the five cultivation dishes 16 on the delivery station 28 after properly positioning the infected explants 12 on them.

An Agrobacterium culture is a widely utilized method for introducing an expression vector into plants is based on the natural transformation system of Agrobacterium. Horsch et al., Science 227:1229 (1985). A. tumefaciens and A. rhizogenes are plant pathogenic soil bacteria known to be useful to genetically transform plant cells. The Ti and Ri plasmids of A. tumefaciens and A. rhizogenes, respectively, carry genes responsible for genetic transformation of the plant. Kado, C. I., Crit. Rev. Plant. Sci. 10:1 (1991). Descriptions of Agrobacterium vector systems and methods for Agrobacterium-mediated gene transfer are also available, for example, Gruber et al., supra, Miki et al., supra, Moloney et al., Plant Cell Reports 8:238 (1989), and U.S. Pat. Nos. 4,940,838 and 5,464,763.

If Agrobacterium is used for the transformation, the DNA to be inserted should be cloned into special plasmids, namely either into an intermediate vector or into a binary vector. Intermediate vectors cannot replicate themselves in Agrobacterium. The intermediate vector can be transferred into Agrobacterium by means of a helper plasmid (conjugation). The Japan Tobacco Superbinary system is an example of such a system (reviewed by Komari et al. (2006) In: Methods in Molecular Biology (K. Wang, ed.) No. 343: Agrobacterium Protocols (2^{nd} Edition, Vol. 1) HUMANA PRESS Inc., Totowa, N.J., pp. 15-41; and Komori et al. (2007) Plant Physiol. 145:1155-1160). Binary vectors can replicate themselves both in E. coli and in Agrobacterium. They comprise a selection marker gene and a linker or polylinker which are framed by the right and left T-DNA border regions. They can be transformed directly into Agrobacterium (Holsters, 1978). The Agrobacterium used as host cell is to comprise a plasmid carrying a vir region. The Ti or Ri plasmid also comprises the vir region necessary for the transfer of the T-DNA. The vir region is necessary for the transfer of the T-DNA into the plant cell. Additional T-DNA may be contained.

The virulence functions of the Agrobacterium host will direct the insertion of a T-strand containing the construct and adjacent marker into the plant cell DNA when the cell is infected by the bacteria using a binary T DNA vector (Bevan (1984) Nuc. Acid Res. 12:8711-8721) or the co-cultivation procedure (Horsch et al. (1985) Science 227:1229-1231). Generally, the Agrobacterium transformation system is used to engineer dicotyledonous plants (Bevan et al. (1982) Ann. Rev. Genet 16:357-384; Rogers et al. (1986) Methods Enzymol. 118:627-641). The Agrobacterium transformation system may also be used to transform, as well as transfer, DNA to monocotyledonous plants and plant cells. See U.S. Pat. No. 5,591,616; Hernalsteen et al. (1984) EMBO J 3:3039-3041; Hooykass-Van Slogteren et al. (1984) Nature 311:763-764; Grimsley et al. (1987) Nature 325:1677-179; Boulton et al. (1989) Plant Mol. Biol. 12:31-40; and Gould et al. (1991) Plant Physiol. 95:426-434.

Split soybean seeds comprising a portion of an embryonic axis may be typically inoculated with a culture of Agrobacterium, e.g., Agrobacterium tumefaciens or Agrobacterium rhizogenes, containing a suitable genetic construct for about 0.5 to 3.0 hours, more typically for about 0.5 hours, followed by a period of co-cultivation on suitable medium for up to about 5 days. Explants that putatively contain a copy of the transgene arise from the culturing of the transformed split soybean seeds comprising a portion of an embryonic axis. These explants may be identified and isolated for further tissue propagation.

A number of alternative techniques can also be used for inserting DNA into a host plant cell. Those techniques include, but are not limited to, transformation with T-DNA delivered by Agrobacterium tumefaciens or Agrobacterium rhizogenes as the transformation agent. From example of Agrobacterium technology are described in, for example, in U.S. Pat. No. 5,177,010, U.S. Pat. No. 5,104,310, European Patent Application No. 0131624B1, European Patent Application No. 120516, European Patent Application No. 159418B1, European Patent Application No. 176112, U.S. Pat. No. 5,149,645, U.S. Pat. No. 5,469,976, U.S. Pat. No. 5,464,763, U.S. Pat. No. 4,940,838, U.S. Pat. No. 4,693,976, European Patent Application No. 116718, European Patent Application No. 290799, European Patent Application No. 320500, European Patent Application No. 604662, European Patent Application No. 627752, European Patent Application No. 0267159, European Patent Application No. 0292435, U.S. Pat. No. 5,231,019, U.S. Pat. No. 5,463,174, U.S. Pat. No. 4,762,785, U.S. Pat. No. 5,004,863, and U.S. Pat. No. 5,159,135. The use of T-DNA-containing vectors for the transformation of plant cells has been intensively researched and sufficiently described in European Patent Application 120516; An et al, (1985, EMBO J. 4:277-284), Fraley et al, (1986, Crit. Rev. Plant Sci. 4: 1-46), and Lee and Gelvin (2008, Plant Physiol. 146: 325-332), and is well established in the field.

Another known method of plant transformation is microprojectile-mediated transformation wherein DNA is carried on the surface of microprojectiles. In this method, the expression vector is introduced into plant tissues with a biolistic device that accelerates the microprojectiles to speeds sufficient to penetrate plant cell walls and membranes. Sanford et al., Part. Sci. Technol. 5:27 (1987), Sanford, J. C., Trends Biotech. 6:299 (1988), Sanford, J. C., Physiol. Plant 79:206 (1990), Klein et al., Biotechnology 10:268 (1992).

Alternatively, gene transfer and transformation methods include, but are not limited to, protoplast transformation through calcium chloride precipitation, polyethylene glycol (PEG)- or electroporation-mediated uptake of naked DNA (see Paszkowski et al. (1984) EMBO J 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505).

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

What is claimed is:

1. A method for automated explant preparation, the method comprising:
operating a pump to fill an explant dish including a plurality of explants with an Agrobacterium solution,
operating a first robotic arm to move the filled explant dish onto a shaker plate of a shaker station,
operating the shaker station to move the shaker plate in a direction within a plane defined by the shaker plate to infect the plurality of explants with the Agrobacterium solution, and
operating a second robotic arm to move an explant from the filled explant dish to a predetermined position on a cultivation media dish in response to determining the explant has been infected with the Agrobacterium solution.

2. The method of claim 1, further comprising operating the first robotic arm to move the cultivation media dish to the delivery station in response to determining the cultivation media dish has a predetermined number of explants positioned on the cultivation media dish.

3. The method of claim 2, wherein determining the cultivation media dish has the predetermined number of explants positioned on the cultivation media dish comprises determining the cultivation media dish has a number (n) of explants positioned on the cultivation media dish and the explants are evenly spaced 360/n degrees apart on the cultivation media dish.

4. The method of claim 2, wherein operating the first robotic arm to move the cultivation media dish comprises:
operating the first robotic arm to secure a lid of the cultivation media dish onto the cultivation media dish, and
operating the first robotic arm to move the secured cultivation media dish to the delivery station.

5. The method of claim 1, further comprising:
capturing an image of a base of the filled explant dish with a camera,
determining a location of an explant in the filled explant dish based on the image, and
operating the second robotic arm to grip the explant at the location,
wherein operating the second robotic arm to move the explant comprises operating the second robotic arm to move the explant in response to operating the second robotic arm to grip the explant.

6. The method of claim 5, wherein determining the location of the explant in the filled explant dish comprises:
determining locations of the plurality of explants in the filled explant dish, and
selecting the explant from the plurality of explants.

7. The method of claim 1, further comprising selecting the cultivation media dish from a plurality of cultivation media dishes based on a number of explants currently positioned on each of the cultivation media dishes.

8. The method of 7, wherein selecting the cultivation media dish comprises selecting a cultivation media dish having fewer than six explants currently positioned on the cultivation media dish, and
wherein operating the second robotic arm to move the explant from the filled explant dish to the predetermined position on the selected cultivation media dish comprises determining a predetermined position on the selected cultivation media dish to which to move the explant based on a position of each other explant currently positioned on the cultivation media dish.

9. The method of claim 1, further comprising operating the first robotic arm to move each cultivation media dish of a plurality of cultivation media dishes from a dish dispenser to a predetermined position on a transfer station different from a position of each other cultivation media dish of the plurality of cultivation media dishes.

10. The method of claim 9, further comprising operating a second pump to pump the Agrobacterium solution from the filled explant dish and into a solution waste container in response to determining each cultivation media dish has a predetermined number of explants positioned on the cultivation media dish.

11. The method of claim 10, further comprising operating the first robotic arm to move the filled explant dish to a dish waste container in response to determining the Agrobacterium solution has been removed from the filled explant dish.

12. The method of claim 1, wherein operating the first robotic arm to move the filled explant dish comprises operating a claw grip of the first robotic arm with a compressed air source to grasp the filled explant dish, and
wherein operating the second robotic arm to move the explant comprises operating the second robotic arm to secure the explant with a suction force applied to the explant with a negative pressure source of the second robotic arm.

13. The method of claim 1, wherein operating the second robotic arm to move the explant comprises operating the second robotic arm to move the explant from the filled explant dish in response to determining that a desired infection time associated with infection of the explant has been reached.

14. The method of claim 1, wherein operating the shaker station to move the plate comprises moving the plate in movement pattern including at least one of rotational or side-to-side movements within the plane defined by the plate.

15. The method of claim 1, further comprising sterilizing a grip of the second robotic arm.

16. The method of claim 1, wherein the Agrobacterium solution comprises Agrobacterium tumefaciens.

17. The method of claim 1, wherein the Agrobacterium solution comprises Agrobacterium rhizogenes.

18. An explant preparation apparatus, comprising:
a first robotic arm including a claw grip to grasp explant dishes for movement,
a second robotic arm including a suction grip to secure explants with suction force for movement,
a pump configured to deliver an Agrobacterium solution,
a shaker station including a shaker plate and configured to move the shaker plate, and
an electronic controller configured to:
operate the pump to fill an explant dish including a plurality of explants with an Agrobacterium solution,
operate the first robotic arm to move the filled explant dish onto a shaker plate of a shaker station,
operate the shaker station to move the shaker plate in a direction within a plane defined by the shaker plate to infect the plurality of explants with the Agrobacterium solution, and
operate the second robotic arm to move an explant from the filled explant dish to a predetermined position on a cultivation media dish in response to a determination that the explant has been infected with the Agrobacterium solution.

19. The explant preparation apparatus of claim 18, further comprising a third robotic arm including a claw grip to grasp explant dishes for movement.

20. The explant preparation apparatus of claim 18, wherein:
the first robotic arm includes a compressed air source, and
the electronic controller is configured to operate the compressed air source to move the claw grip between an open and closed position.

21. The explant preparation apparatus of claim 18, wherein the Agrobacterium solution comprises Agrobacterium tumefaciens.

22. The explant preparation apparatus of claim 18, wherein the Agrobacterium solution comprises Agrobacterium rhizogenes.

23. A dish dispensing system, comprising:
a housing,
an elongated body secured to the housing and centered about a longitudinal axis, wherein the elongated body is configured to secure a stack of petri dishes along the longitudinal axis,
a first pneumatic device positioned in the housing and configured to move a set of petri dishes of the stack of petri dishes along the longitudinal axis in a first direction to separate a first petri dish of the stack of petri dishes from the set of petri dishes, and
a second pneumatic device positioned in the housing and configured to move the separated first petri dish along an axis orthogonal to the longitudinal axis.

24. The dish dispensing system of claim 23, wherein the first pneumatic device comprises a pair of dish gripping arms configured to secure a bottom petri dish of the set of petri dishes.

25. The dish dispensing system of claim 23, wherein the second pneumatic device is configured to move the separated first petri dish to a location outside the housing.

26. The dish dispensing system of claim 23, further comprising a third pneumatic device positioned in the housing and configured to move the set of petri dishes in a second direction opposite the first direction in response to a determination that the separated first petri dish has been removed from a plate extender operated by the second pneumatic device.
